(19) United States
(12) Patent Application Publication
(10) Pub. No.: US 2016/0067158 A1
(43) Pub. Date: Mar. 10, 2016

(54) AGENTS CONTAINING LARGE QUANTITIES OF UV STABILIZERS

(71) Applicant: BASF SE, Ludwigshafen (DE)

(72) Inventors: Matthias HLOUCHA, Koeln (DE); Esther KUESTERS, Erkrath (DE); Stephanie ACKER, Dietwiller (FR); Heike FLOESSER-MUELLER, Brasschaat (BE); Jasmin SCHORB, Monheim (DE)

(73) Assignee: BASF SE, Ludwigshafen (DE)

(21) Appl. No.: 14/890,307

(22) PCT Filed: May 6, 2014
(86) PCT No.: PCT/EP14/59187

§ 371 Date: Nov. 10, 2015

(30) Foreign Application Priority Data
May 10, 2013 (DE) 13167285.9

(51) Int. Cl. A61K 8/33; A61K 8/41; A61K 8/49; A61Q 17/04; A61K 8/37

(52) U.S. Cl. CPC A61K 8/33; A61Q 17/04; A61K 8/37; A61K 8/4966; A61K 8/415; A61K 2800/596

(57) ABSTRACT

The invention relates to highly concentrated agents for the preparation of sunscreen final formulations, comprising
(a) 15 to 50 wt. % of oily substance,
(b) 4 to 30 wt. % of anionic surfactant,
(c) 0.5 to 25 wt. % of further co-surfactant which differs from (b),
(d) 25 to 75 wt. % of UV filter, and
(e) optionally water and/or auxiliary substances to 100 wt. %.
   and its use for the preparation of sunscreen final formulations and the preparation thereof.

The present invention relates to agents for the preparation of highly concentrated formulations comprising large amounts of UV filters, a process for the preparation of sunscreen final formulations based on such agents and the use of such agents for the preparation of cosmetic formulations having extremely high sun protection factors (SPF up to 50+).

From the dermatological aspect, in addition to the care effect for the skin sunscreen formulations above all should reduce or as far as possible prevent the adverse effects of solar radiation. For this, sunscreen formulations comprise UV filters which via their activity, amount and combination determine the UV protections, usually expressed as the SPF factor.

For the manufacturer of such sunscreen formulations it is important for the UV filters to be present in a form which has a shelf life, which can be converted into their own, defined sunscreen final formulations easily and as far as possible at room temperature. By simple blending the manufacturer would like to obtain an O/W care emulsion of which the SPF factor can be determined according to his own requirements.

The laid-open specification DE 102005011785 discloses cosmetic O/W emulsions which can also comprise light protection filters. These O/W emulsions are distinguished by an emulsifier combination of non-carbohydrate polyol partial esters of fatty acids, in particular polyglycerol partial esters, and emulsifiers based on carbohydrates, in particular glycosides. With the aid of these specific emulsifier combinations emulsion concentrates are first prepared, which must then be diluted rapidly with water, since they do not have a shelf life.

The object of the present invention is to provide agents having large amounts of UV filters which have a shelf life and thus can also first be converted into O/W emulsions with water after weeks. It should be possible to employ the agents as concentrates, i.e. large amounts of UV filter should be dissolved in large amounts of oils without crystallization of these UV filters occurring during a relatively long storage period. It is also desirable for these agents in the sense of a concentrate to comprise as little water as possible. Furthermore, it should be possible to convert the agents into O/W emulsions for sun protection easily and without the particular presence of shearing forces.

Surprisingly, it has been possible to achieve the object by highly concentrated agents for the preparation of sunscreen final formulations, comprising
(a) 15 to 50 wt. % of oily substance,
(b) 4 to 30 wt. % of anionic surfactant,
(c) 0.5 to 25 wt. % of further co-surfactant which differs from (b),
(d) 25 to 75 wt. % of at least one UV filter, and
(e) optionally water and/or auxiliary substances to 100 wt. %.

The agents according to the invention have a shelf life and show no crystallizing out or sediment of UV filters even after a relatively long storage period. Water can be added to the agents without problems, as a result of which very finely divided sunscreen final formulations of the O/W emulsion type are formed, which in turn also have a very good shelf life and are finely divided homogeneously. Furthermore, the agents in the sense of a concentrate are suitable for giving the user free design possibilities for establishing the desired SPF factor of the sunscreen final formulation.

In the context of the present invention, the SPF factor (sun protection factor, SPF) serves to evaluate light protection preparations (sun creams) on humans (in vivo). It indicates how much longer a person with a sunscreen agent can be exposed to the sun without suffering sunburn than would be possible with the particular individual's self-protection time.

The SPF is determined by the “COLIPA International Sun Protection Factor Test Method” (COLIPA, May 2006), the increase in the skin reddening threshold (minimal erythema dose, MED) after standardized application of light protection preparations being determined as a function of the irradiation time.

In the context of the present invention, possible oily substances (a) are, for example, Guerbet alcohols based on fatty alcohols having 6 to 18, preferably 8 to 10 carbon atoms (e.g. Eutanol® G), esters of linear C₆-C₂₂-fatty acids with linear or branched C₆-C₂₂-fatty alcohols and esters of branched C₆-C₁₃-carboxylic acids with linear or branched C₆-C₂₂-fatty alcohols, such as e.g. myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. In addition, esters of linear C₆-C₂₂-fatty acids with branched alcohols, in particular 2-ethylhexanol, esters of C₃-C₃₈-alkylhydroxycarboxylic acids with linear or branched C₆-C₂₂-fatty alcohols, in particular diethylhexyl malate, esters of linear and/or branched fatty acids with polyhydric alcohols (such as e.g. propylene glycol, dimer diol or trimer triol) and/or Guerbet alcohols, triglycerides based on C₆-C₁₀-fatty acids, liquid mono/di/triglyceride mixtures based on C₆-C₁₈-fatty acids, esters of C₆-C₂₂-fatty alcohols and/or Guerbet alcohols with aromatic carboxylic acids, in particular benzoic acid, esters of C₂-C₁₂-dicarboxylic acids with linear or branched alcohols having 1 to 22 carbon atoms or polyols having 2 to 10 carbon atoms and 2 to 6 hydroxyl groups, plant oils, branched primary alcohols, substituted cyclohexanes, linear and branched C₆-C₂₂-fatty alcohol carbonates, such as e.g. Dicaprylyl Carbonate (Cetiol® OE), Guerbet carbonates based on fatty alcohols having 6 to 18 preferably 8 to 10 C atoms, esters of benzoic acid with linear and/or branched C₆-C₂₂-alcohols (e.g. Finsolv® TN), linear or branched, symmetric or unsymmetric dialkyl ethers having 6 to 22 carbon atoms per alkyl group, such as e.g. Dicaprylyl Ether (Cetiol® OE), ring-opening products of epoxidized fatty acid esters with polyols (Hydagen® HSP, Sovermol® 750, Sovermol® 1102), silicone oils (cyclomethicone, silicon methicone types and others) and/or aliphatic or naphthenic hydrocarbons, such as e.g. mineral oil, Vaseline, petrolatum, squalane, squalene, isohexadecane or dialkylcyclohexanes are suitable in consideration.

Preferred oily substances (a) are medium-polarity oils, in particular esters of C₂-C₁₂-dicarboxylic acids with linear or branched alcohols having 1 to 22 carbon atoms and/or linear and branched C₆-C₂₂-fatty alcohol carbonates Adipic acid esters of linear or branched alcohols having 1 to 22 carbon atoms, very particularly of linear alcohols having 1 to 6 carbon atoms, are particularly suitable here.

Linear and branched fatty alcohol carbonates, in particular Dicaprylyl Carbonate, are particularly preferably used as oily substances (a).

Outstanding results are achieved with dibutyl adipate.

Preferably, the oily substances are present in an amount of 20-35 wt. %, based on the agents according to the invention.

The anionic surfactants (b) are characterized by one or more anionic group which confers solubility in water, such as e.g. a carboxylate, sulfate, sulfonate or phosphate group, and a lipophilic radical. In addition the molecule can contain polyglycol ether, ester, ether and hydroxyl groups. Anionic surfactants which are tolerated by skin are known to the person skilled in the art in large numbers from relevant handbooks and are commercially obtainable.

Examples of preferred anionic surfactants are, in each case in the form of their salts, ether-carboxylic acids, acylsarcosides having 8 to 24 C atoms in the acyl group, acyltaurides having 8 to 24 C atoms in the acyl group, acylisethionates having 8 to 24 C atoms in the acyl group, sulfosuccinic acid mono- and dialkyl esters having 8 to 24 C atoms in the alkyl group and sulfosuccinic acid monoalkyl polyoxyethyl esters having 8 to 24 C atoms in the alkyl group and 1 to 6 oxyethyl groups, linear alkanesulfonates having 8 to 24 C atoms, linear alpha-olefinsulfonates having 8 to 24 C atoms, alpha-sulfo-fatty acid methyl esters of fatty acids having 8 to 30 C atoms, alkyl sulfates, alkyl polyglycol ether sulfates, esters of tartaric acid and citric acid, alkyl and/or alkenyl ether phosphates, sulfated fatty acid alkylene glycol esters, monoglyceride sulfates and monoglyceride ether sulfates as well as condensation products of C₃-C₃₀-fatty alcohols with protein hydrolysates and/or amino acids and derivatives thereof, so-called protein fatty acid condensates, e.g. Lamepon®, Gluadin®, Hostapon® KCG or Amisoft®.

The salts of these surfactants are preferably selected from the sodium, potassium and ammonium and the mono-, di- and trialkanalammonium salts having 2 to 4 C atoms in the alkanol group.

Particularly suitable anionic surfactants are liquid at room temperature, preferably from 18 to 25° C. A desirable feature in particular of these anionic surfactants is that they have a low water content of at most 10 wt. %, preferably 0.1 to 5 wt. %, based on the anionic surfactant.

Outstandingly suitable surfactants in this context are alk(en)yl polyglycol ether citrates and in particular mixtures of mono-, di- and triesters of citric acid and alkoxylated alcohols which correspond to the formula (I):

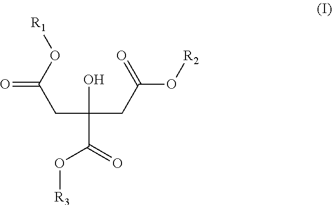

wherein
R₁, R₂ and R₃ independently of each other denote hydrogen or the radical of the formula

R₄(OCH₂CHR₅)ₙ (II)
wherein
R₄ represents a linear or branched alkyl and/or alkenyl radical having 6 to 22 carbon atoms,
R₅ represents hydrogen or a methyl radical and
n represents a number from 1 to 20, with the condition that at least one of the radicals R₁, R₂ or R₃ is other than hydrogen.

Typical examples of the alcohol part of the esters are addition products of on average 1 to 20 mol, preferably 5 to 10 mol of ethylene oxide and/or propylene oxide on caproyl alcohol, capryl alcohol, 2-ethylhexyl alcohol, capric alcohol, lauryl alcohol, isotridecyl alcohol, myristyl alcohol, cetyl alcohol, palmitolelyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol and technical grade mixtures thereof.

It is particularly preferable to use alkyl polyalkylene glycol ether citrates based on addition products of from 5 to 10, in particular approximately 7 mol of ethylene oxide on technical grade C₁₂-C₁₈-, in particular C₁₂-C₁₄-fatty alcohol fractions. The polyethylene glycol ethers of lauryl alcohol, laureth-7 citrates, which are obtainable, for example, under the name Plantapon® LC7 (BASF & Personal Care & Nutrition GmbH), are very particularly preferred.

Such alk(en)yl polyglycol ether citrates are advantageous for the agents according to the invention since they are liquid anionic surfactants having a low water content of max. 5 wt. %, based on the anionic surfactant.

The anionic surfactants (b) are preferably present in amounts of from 7 to 17 wt. %, based on the agents according to the invention.

The agents according to the invention furthermore comprise at least (c) 0.5 to 25 wt. % of a further co-surfactant which differs from (b).

Suitable co-surfactants are, in principle, zwitter-ionic, ampholytic, cationic and/or nonionic surfactants.

Those surface-active compounds which carry at least one quaternary ammonium group and at least one —COO(−) or —SO₃(−) group in the molecule are called zwitter-ionic surfactants. Particularly suitable zwitter-ionic surfactants are the so-called betaines, such as the N-alkyl-N,N-dimethylammonium glycinates, for example coco-alkyldimethylammonium glycinate, N-acylaminopropyl-N, N-dimethylammonium glycinates, for example coco-acylamimopropyl-dimethylammonium glycinate, and 2-alkyl-3-carboxymethyl-3-hydroxyethylimidazoline having in each case 8 to 18 C atoms in the alkyl or acyl group, and coco-acylaminoethyl-hydroxyethylcarboxymethyl glycinate. The fatty acid amide derivative known under the INCI name Cocamidopropyl Betaine is a preferred zwitter-ionic surfactant. Tego® Betain 810 (INCI: Capryl/Capramidopropyl Betaine) and a surfactant mixture of Rewopol® SBCS 50K (INCI: Disodium PEG-5 Laurylcitrate Sulfosuccinate, Sodium Laureth Sulfate) and Tego® Betain 810 (Capryl/Capramidopropyl Betaine), in particular in the weight ratio of 1:4 to 4:1, very particularly preferably in the weight ratio of from 1:4 to 1:1, are particularly preferred according to the invention.

Ampholytic surfactants are understood as meaning those surface-active compounds which contain, apart from a C₃-C₁₈-alkyl or acyl group, at least one free amino group and at least one —COOH or —SO₃H group in the molecule and are capable of formation of inner salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 C atoms in the alkyl group. Preferred ampholytic surfactants are N-coco-alkylaminopropionate, coco-acylaminoethyl-aminopropionate and C₁₂₋₁₈-acylsarcosine.

Quaternary ammonium compounds in particular can be used as cationic surfactants. Surfactants from this substance class have a particularly high affinity for the skin and can improve the degree of sensory smoothness. These include, inter alia, ammonium halides, in particular chlorides and bromides, such as alkyltrimethylammonium chlorides, dialkyldimethylammonium chlorides and trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride and tricetylmethylammonium chloride. The very readily biodegradable quaternary ester compounds, such as, for example, the dialkylammonium methosulfates and methylhydroxyalkyldialkoyloxyalkylammonium methosulfates marketed under the trade name Stepantex® and the corresponding products of the Dehyquart® series, can furthermore be employed as cationic surfactants. The term “esterquats” is in general understood as meaning quaternized fatty acid triethanolamine ester salts. They impart to the compositions particularly soft feel. These are known substances which are prepared by the relevant methods of organic chemistry. Further cationic surfactants which can be used according to the invention are the quaternized protein hydrolysates.

Nonionic surfactants are particularly preferably present as co-surfactants, for example
addition products of from 2 to 50 mol of ethylene oxide and/or 0 to 20 mol of propylene oxide on linear fatty alcohols having 8 to 40 C atoms, on fatty acids having 12 to 40 C atoms and on alkylphenols having 8 to 15 C atoms in the alkyl group;
C_{12/18}-fatty acid mono- and diesters of addition products of from 1 to 50 mol of ethylene oxide on glycerol; glycerol mono- and diesters and sorbitan mono- and diesters of saturated and unsaturated fatty acids having 6 to 22 carbon atoms and ethylene oxide addition products thereof,
alkyl mono- and oligoglycoside having 8 to 22 carbon atoms in the alkyl radical and ethoxylated analogues thereof; addition products of from 7 to 60 mol of ethylene oxide on castor oil and/or hydrogenated castor oil;
polyol and/or polyglycerol esters, such as e.g. polyglycerol diisostearate or polyglycerol dimerate or polyglycerol 12-hydroxystearate;
addition products of from 2 to 15 mol of ethylene oxide on castor oil and/or hydrogenated castor oil;
partial esters based on linear, branched, unsaturated or saturated C₆-C₂₂-fatty acids, ricinoleic acid and 12-hydroxystearic acid with, pentaerythritol, dipentaerythritol, sugar alcohols (e.g. sorbitol), alkyl glucosides (e.g. methyl glucoside, butyl glucoside, lauryl glucoside) and polyglucosides (e.g. cellulose), or mixed esters, such as e.g. glyceryl stearate citrate and glyceryl stearate lactate;
wool wax alcohols;
polysiloxane/polyalkyl polyether copolymers and corresponding derivatives;
mixed esters of pentaerythritol, fatty acids, citric acid and fatty alcohol and/or mixed esters of fatty acids having 6 to 22 carbon atoms, methylglucose and polyols, preferably glycerol or polyglycerol; and
polyalkylene glycols.

The addition products of ethylene oxide and/or of propylene oxide on fatty alcohols, fatty acids, alkylphenols, glycerol mono- and diesters and sorbitan mono- and diesters of fatty acids or on castor oil are known, commercially obtainable products. These are homologue mixtures, the average degree of alkoxylation of which corresponds to the ratio of the substance amounts of ethylene oxide and/or propylene oxide and substrate with which the addition reaction is carried out. They are W/O or O/W emulsifiers, depending on the degree of ethoxylation. For the preparations according to the invention, the reaction products with 1-100 mol of ethylene oxide are particularly suitable.

Advantageous compounds from the group of nonionic surfactants are partial esters of polyols, in particular of C₃-C₆-polyols, such as, for example, glyceryl monoesters, partial esters of pentaerythritol or sugar esters, e.g. sucrose distearate, sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical grade mixtures thereof. Addition products of from 1 to 30, preferably 5 to 10 mol of ethylene oxide on the sorbitan esters mentioned are also suitable nonionic surfactants.

Nonionic surfactants from the group of alkyl oligoglycosides are particularly skin-friendly and may therefore preferably be suitable in the context of the invention. C₃-C₂₂-alkyl mono- and oligoglycosides, their preparation and their use are known from the prior art. Their preparation is carried out in particular by reaction of glucose or oligosaccharides with primary alcohols having 8 to 22 C atoms, preferably 12 to 22, and particularly preferably 12 to 18 C atoms. With respect to the glycoside radical, both monoglycosides in which a cyclic sugar residue is bonded glycosidically to the fatty alcohol and oligomeric glycosides having a degree of oligomerization of up to preferably about 8 are suitable. The degree of oligomerization here is a statistical mean based on a conventional distribution of homologues for such technical grade products Products which are available under the name Plantacare® contain a glucosidically bonded C₃-C₁₆-alkyl group on an oligoglucoside radical, the average degree of oligomerization of which is 1 to 2. The acylglucamides derived from glucamine are also suitable as nonionic surfactants.

Nonionic surfactants, preferably polyol and/or polyglycerol esters, are very particularly preferably present as co-surfactants in the agents according to the invention as component (c), and/or alkyl oligoglycosides.

The polyol component of these surfactants can be derived from substances which have at least two, preferably 3 to 12 and in particular 3 to 8 hydroxyl groups and 2 to 12 carbon atoms. Typical examples are:
glycerol and polyglycerol;
alkylene glycols, such as, for example, ethylene glycol, diethylene glycol, propylene glycol;
methylol compounds, such as, in particular, trimethylolethane, trimethylolpropane, trimethylolbutane, pentaerythritol and dipentaerythritol;
alkyl oligoglucosides having 1 to 22, preferably 1 to 8 and in particular 1 to 4 carbons in the alkyl radical, such as, for example, methyl and butyl glucoside;
sugar alcohols having 5 to 12 carbon atoms, such as, for example, sorbitol or mannitol;
sugars having 5 to 12 carbon atoms, such as, for example, glucose or sucrose;
amino-sugars, such as, for example, glucamine.

Reaction products based on polyglycerol are of particular importance because of their excellent use properties.

The acid component of these surfactants can be derived from straight-chain, branched, saturated and/or unsaturated carboxylic acids, optionally with functional groups, such as hydroxyl groups. The acid component is particularly preferably fatty acids having 12 to 22 carbon atoms, which optionally carry a hydroxyl group, and in particular hydroxystearic acid.

Reaction product of poly-12-hydroxystearic acid with polyglycerols of the following distribution of homologues have proved to be particularly advantageous as nonionic surfactants (preferred amounts are stated in parentheses):
Glycerols: 5 to 15 (15 to 30) wt. %
Diglycerols: 15 to 40 (20 to 32) wt. %
Triglycerols: 10 to 15 (15 to 25) wt. %
Tetraglycerols: 5 to 20 (8 to 15) wt. %
Pentaglycerols: 2 to 10 (3 to 8) wt. %
Oligoglycerols: to 100 wt. %

In a preferred embodiment of the invention the diester of polyhydroxystearic acid, polyglyceryl 2-dipolyhydroxystearate, which is marketed, for example, by BASF Personal Care and Nutrition GmbH under the name Dehymuls® PGPH, is used as a glyceryl ester.

In the agents according to the invention the further co-surfactants are conventionally present in an amount of 0.5-25 wt. %; an amount of 3.0-18 wt. % is preferred, and in particular 7-18 wt. %.

Finally, the agents according to the invention comprise large amounts, i.e. in the range of from 25 to 75 wt. %, preferably 35 to 65 wt. %, in particular 40 to 60 wt. % of UV filters (d).

The following substance classes are preferably employed according to the invention as UV filters corresponding to component (d) (INCI names):
(d₁) p-aminobenzoic acid derivatives,
(d₂) salicylic acid derivatives,
(d₃) benzophenone derivatives,
(d₄) dibenzoylmethane derivatives,
(d₅) diphenyl acrylates,
(d₆) 3-imidazol-4-yl-acrylic acid and its esters;
(d₇) benzofuran derivatives;
(d₈) polymeric UV absorbers;
(d₉) cinnamic acid derivatives;
(d₁₀) camphor derivatives;
(d₁₁) hydroxyphenyltriazine derivatives;
(d₁₂) benzotriazole derivatives;
(d₁₃) trianilino-s-triazine derivatives;
(d₁₄) 2-phenylbenzimidazole-5-sulfonic acid and salts thereof;
(d₁₅) menthyl o-aminobenzoates;
(d₁₆) homosalates;
(d₁₇) tris-biphenyltriazine derivatives;
(d₁₈) TiO₂ (partly encapsulated), ZnO and mica;
(d₁₉) benzylidenemalonates;
(d₂₀) merocyanine derivatives;
(d₂₁) phenylene bis diphenyltriazines;
(d₂₂) imidazoline derivatives; and
(d₂₃) diarylbutadiene derivatives.

Compounds which can be employed by way of example for p-aminobenzoic acid derivatives (d₁) are
4-aminobenzoic acid (PABA); ethyldihydroxypropyl-PABA of the formula

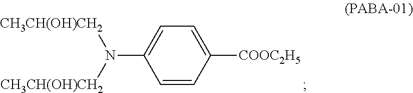

PEG-25-PABA of the formula

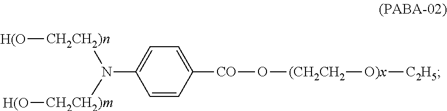

wherein m, n and x have the same meaning and each denote at most 25; octyldimethyl PABA of the formula

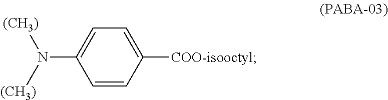

or glycyl aminobenzoate of the formula

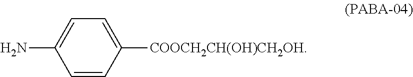

Compounds which can be employed by way of example for salicylic acid derivatives (d₂) are homomenthyl salicylate of the formula

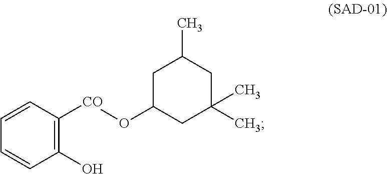 [SMILES: CC1CC(OC(=O)c2ccccc2O)CC(C)(C)C1]

triethanolamine salicylate of the formula

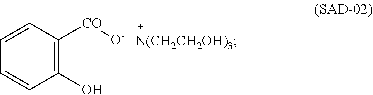

amyl p-dimethylaminobenzoate of the formula

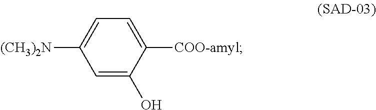

octyl salicylate of the formula

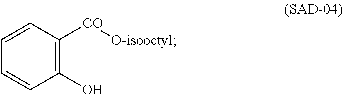

or 4-isopropylbenzyl salicylate of the formula

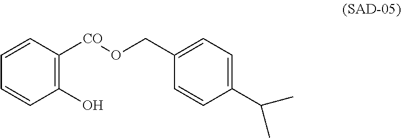 [SMILES: CC(C)c1ccc(COC(=O)c2ccccc2O)cc1]

Compounds which can be employed by way of example for benzophenone derivatives (d₃) are:
benzophenone-3-(2-hydroxy-4-methoxybenzophenone); benzophenone-4-(2-hydroxy-4-methoxybenzophenone-5-sulfonic acid); benzophenone-8-(2,2′-dihydroxy-4-methoxybenzophenone); or amino-substituted hydroxybenzophenones of the formula

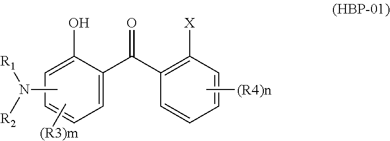

wherein
R₁ and R₂ denote hydrogen, C₁-C₂₀-alkyl, C₂-C₁₀-alkenyl, C₃-C₁₀-cycloalkyl, C₃-C₁₀-cycloalkenyl, wherein the substituents R₁ and R₂ together with the nitrogen atom to which they are bonded can form a 5- or 6-membered ring;
R₃ and R₄ independently of each other denote C₁-C₂₀-alkyl; C₂-C₁₀-alkenyl; C₃-C₁₀-cycloalkyl; C₃-C₁₀-cycloalkenyl; C₁-C₂₂-alkoxy; C₁-C₂₀-alkoxycarbonyl; C₁-C₁₂-alkylamino; C₁-C₁₂-dialkylamino; optionally substituted aryl; hetaryl; substituents conferring solubility in water selected from the group consisting of a nitrile group, and carboxylate, sulfonate or ammonium radicals;
X denotes hydrogen; COOR₅; or CONR₆R₇;
R₅, R₆, R₇ independently of each other denote hydrogen; C₁-C₂₀-alkyl; C₂-C₁₀-alkenyl; C₃-C₁₀-cycloalkyl; C₃-C₁₀-cycloalkenyl; (Y—O)ₒ—Z; or aryl;
Z denotes —CH₂—CH₃; —CH₂—CH₂—CH₃; —CH₂—CH₂—CH₂—CH₃; or —CH(CH₃)—CH₃;
m denotes 0 to 3;
n denotes 0 to 4; and
o denotes 1 to 20.

Diethylamino Hydroxybenzoyl Hexyl Benzoate corresponding to the formula

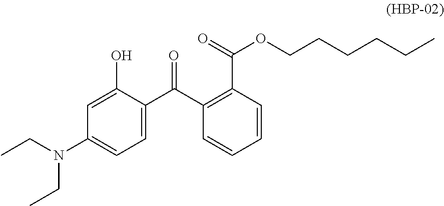 [SMILES: CCCCCCOC(=O)c1ccccc1C(=O)c1ccc(N(CC)CC)cc1O]

is particularly preferred.

Dimeric benzophenone derivatives corresponding to the formula

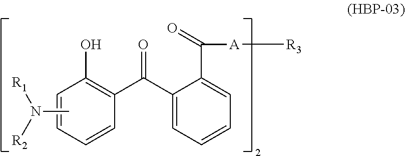

wherein
R₁ and R₂ independently of each other denote C₁-C₂₀-alkyl; C₂-C₂₀-alkenyl; C₃-C₁₀-cycloalkyl; C₃-C₁₀-cycloalkenyl; or R₁ and R₂ together with the nitrogen atom to which they are bonded form a 5- or 6-membered heterocyclic ring;
R₃ denotes alkylene, cycloalkylene, alkenylene or phenylene optionally substituted by a carbonyl or carboxyl group; a biradical of the formula (HBP-03a) *—CH₂—C≡C—CH₂—*; or R₃ together with A forms a bivalent radical of the formula

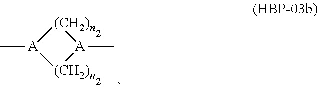

wherein
n₂ denotes a number from 1 to 3;
A denotes —O—; or —N(R₅)—; and
R₅ denotes hydrogen; C₁-C₅-alkyl; or hydroxy-C₁-C₅-alkyl;
can also be employed according to the invention.

In particular, dimeric benzophenone derivatives of the formula

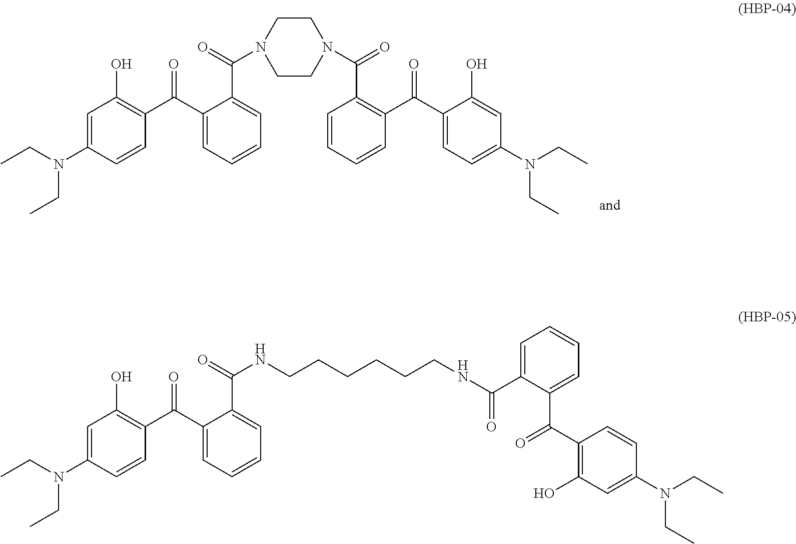

can preferably be employed as UV filters (d₃).

Examples of dibenzoylmethane derivatives (d₄) which can be employed according to the invention are butylmethoxydibenzoylmethane-[1-(4-tert-butylphenyl)-3-(4-methoxyphenyl)propane-1,3-dione].

Examples of diphenylacrylate derivatives (d₅) which can be employed according to the invention are octocrylene-(2-ethylhexyl 2-cyano-3,3′-diphenylacrylate) or etocrylene (ethyl 2-cyano-3,3′-diphenylacrylate).

Examples of benzofuran derivatives (d₇) which can be employed according to the invention are 3-(benzofuranyl) 2-cyanoacrylate, 2-(2-benzofuranyl)-5-tert-butylbenzoxazole or 2-(p-aminophenyl)benzofuran and in particular the compounds of the formula

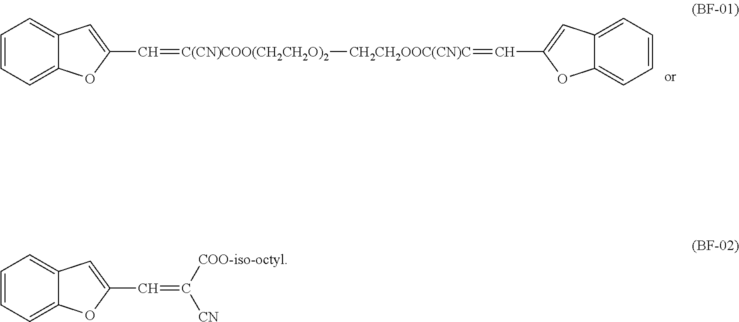

Examples of polymeric UV absorbers (d₈) which can be employed according to the invention and contain one or more organosilicon radicals are benzylidenemalonate derivatives, in particular the compound of the formula

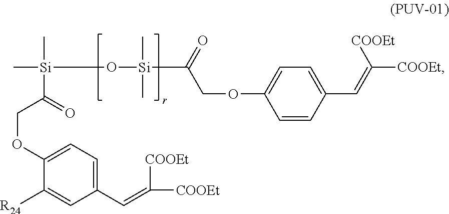

wherein R₂₄ denotes hydrogen or methoxy and r denotes approximately 7; the compound of the formula

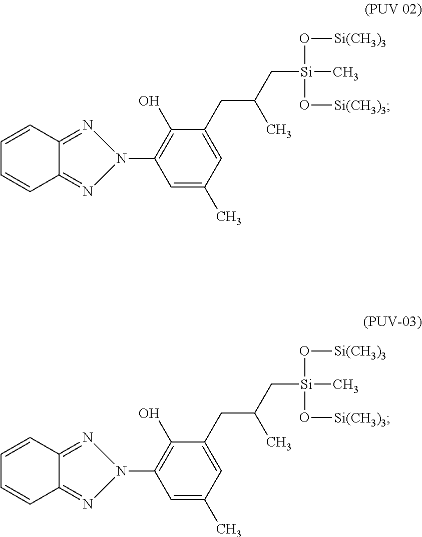

or polysilicone-15 corresponding to the formula

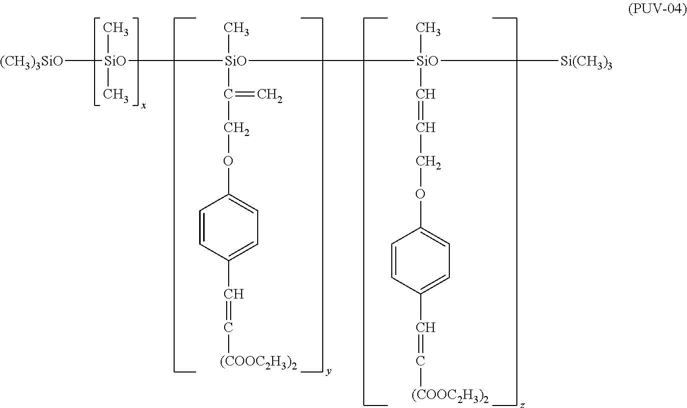

Examples of cinnamic acid esters (d₉) which can be employed according to the invention are octyl methoxycinnamate (4-methoxycinnamic acid 2-ethylhexyl ester), diethanolamine methoxycinnamate (diethanolamine salt of 4-methoxycinnamic acid), isoamyl p-methoxycinnamate (4-ethoxycinnamic acid 2-isoamyl ester), 2,5-diisopropyl methycinnamate or a cinnamic acid amido derivative.

Examples of camphor derivatives (d₁₀) which can be used according to the invention are 4-methylbenzylidenecamphor[3-(4′-methyl)benzylidenebornan-2-one], 3-benzylidenecamphor (3-benzylidenebornan-2-one), polyacrylamidomethylbenzylidenecamphor {N-[2(and 4)-2-oxyborn-3-ylidenemethyl)benzyl]acrylamide polymer}, trimoniumbenzylidenecamphor sulfate-[3-(4′-trimethylammonium)-benzylidenebornan-2-one methylsulfate], terephthalydenedicamphorsulfonic acid {3,3′-(1,4-phenylenedimethine)-bis-(7,7-dimethyl-2-oxobicyclo-[2.2.1]heptane-1-methanesulfonic acid} or salts thereof, or benzylidenecamphorsulfonic acid [3-(4′-sulfo)benzylidenebornan-2-one] or salts thereof.

Examples of hydroxyphenyltriazine derivatives (d₁₁) which can be used according to the invention are, in particular, bis-resorcinyltriazines of the formula

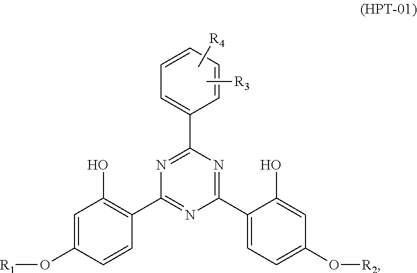

wherein
R₁ and R₂ independently of each other denote hydrogen; C₁-C₁₃-alkyl; C₂-C₁₃-alkenyl; a radical of the formula —CH₂—CH(—OH)—CH₂—O-T₁; a radical of the formula

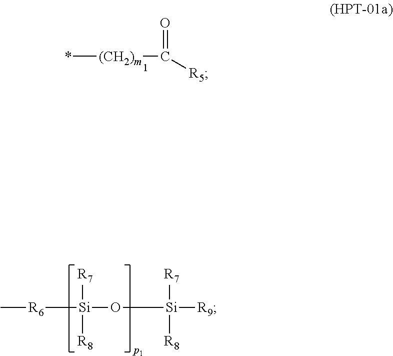

or a radical of the formula (HPT-01h)
R₃, R₅ and R₅ independently of each other denote hydroxyl; C₁-C₅-alkoxy which is unsubstituted or substituted by one or more OH groups; amino; mono- or di-C₁-C₅-alkylamino; M; a radical of the formula

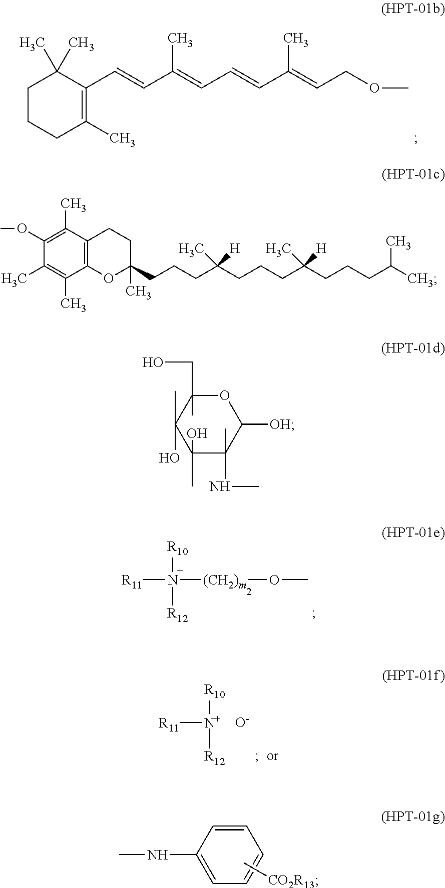

R₁₀, R₁₁ and R₁₂ independently of each other denote C₁-C₁₄-alkyl which is unsubstituted or substituted by one or more OH groups;
R₁₃ denotes hydrogen; M; C₁-C₅-alkyl; or a radical of the formula —(CH₂)ₘ₃—O-T₁;
R₆ denotes the direct bond; a straight-chain or branched C₁-C₄-alkylene radical; or a radical of the formula —Cₘ₄H₂ₘ₄, or —Cₘ₄H₂ₘ₄—O—,
R₇, R₅, and R₉ independently of each other denote C₁-C₁₃-alkyl; C₁-C₁₃-alkoxy or a radical of the formula

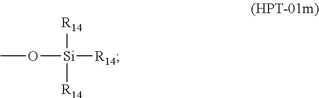

R₁₄ denotes C₁-C₅-alkyl;
M denotes a metal cation;
T₁ denotes hydrogen; or (C₁-C₈-alkyl;
m₁, m₂ and m₃ independently of each other denote 1 to 3;
m₄ denotes 2 to 14; and
p₁ denotes 0 or a number from 1 to 5.

Examples of representatives of the compound class (d₁₁) which may be mentioned are:
2-(4′-methoxyphenyl)-4,6-bis(2′-hydroxy-4′-n-octyloxyphenyl)-1,3,5-triazine;
2,4-bis{[4-(3-(2-propyloxy)-2-hydroxypropyloxy)-2-hydroxy]phenyl}-6-(4-methoxyphenyl)-1,3,5-triazine;
2,4-bis{[4-(2-ethylhexyloxy)-2-hydroxy]phenyl}-6-[4-(2-methoxyethylcarboxyl)-phenylamino]-1,3,5-triazine;
2,4-bis{[4-(tris(trimethylsiloxysilylpropyloxy)-2-hydroxy]phenyl}-6-(4-methoxyphenyl)-1,3,5-triazine;
2,4-bis{[4-(2″methylpropenyloxy)-2-hydroxy]phenyl}-6-(4-methoxyphenyl)-1,3,5-triazine;
2,4-bis{[4-(1′,1′,1′,3′,5′,5′,5′-heptamethyltrisilyl-2″-methylpropyloxy)-2-hydroxy]phenyl}-6-(4-methoxyphenyl)-1,3,5-triazine;
2,4-bis{[4-(3-(2-propyloxy)-2-hydroxypropyloxy)-2-hydroxy]phenyl}-6-[4-ethylcarboxyl)-phenylamino]-1,3,5-triazine;
2,4-bis{[4-(2-ethylhexyloxy)-2-hydroxy]phenyl}-6-(1-methylpyrrol-2-yl)-1,3,5-triazine; or
2,2′-[6-(4-methoxyphenyl)-1,3,5-triazine-2,4-diyl]bis[5-[(2-ethylhexyl)oxy]-(Bis-Ethylhexyloxyphenol Methoxyphenyl Triazine) corresponding to the formula

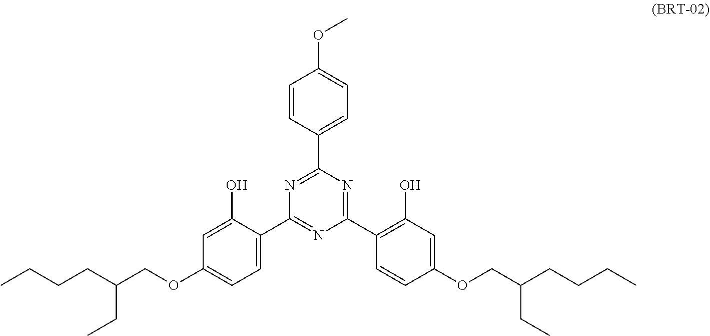 [SMILES: CCCCC(CC)COc1ccc(-c2nc(-c3ccc(OC)cc3)nc(-c3ccc(OCC(CC)CCCC)cc3O)n2)c(O)c1]

Examples of benzotriazole derivatives (d₁₂) which can be used according to the invention correspond to the formula

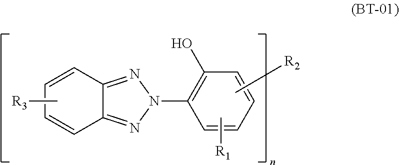

wherein
R₁ denotes hydrogen; C₁-C₁₂-alkyl; C₁-C₁₂-alkoxy; C₁-C₁₂-alkoxycarbonyl; C₅-C₁₀-cycloalkyl or —SO₃M;
R₃ denotes hydrogen; C₁-C₁₈-alkyl; C₁-C₁₂-alkoxy; or halogen; and
n denotes 1 or 2;
if n=1
R₂ denotes C₁-C₂₀-alkyl; C₅-C₁₀-cyclo-C₁-C₅-alkyl; C₁-C₁₂-alkoxy-C₁-C₅-alkyl; C₅-C₁₀-cycloalkoxy-C₁-C₅-alkyl; C₆-C₁₀-aryl; C₆-C₁₀-aryl-C₁-C₅-alkyl;
if n=2
R₂ denotes the direct bond; or —(CH₂)ₚ—; and
p is an integer from 1 to 3.

Preferably, compounds of the formula (BT-01), wherein
R₁ denotes C₁-C₁₂-alkyl; or —SO₃M;
R₃ denotes hydrogen; halogen, preferably Cl;
n denotes 1;
R₂ C₁-C₁₂-alkyl; and
p denotes 1 to 3;
are possible.

Very particularly preferred compounds are those of the formula

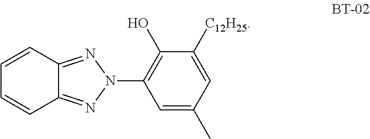

Furthermore, preferred UV filters of the formula BT-01 are those wherein
R₁ denotes hydrogen;
R₃ denotes C₁-C₁₃-alkyl;
n=2; and
R₂ denotes —CH₂—.

Very particularly preferred compounds are those of the formula

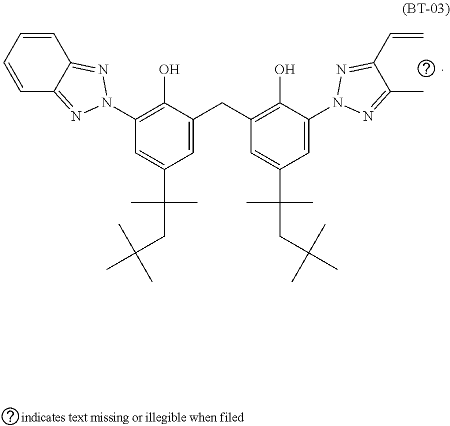 [SMILES: C=Cc1nn(-c2cc(C(C)(C)CC(C)(C)C)cc(Cc3cc(C(C)(C)CC(C)(C)C)cc(-n4nc5ccccc5n4)c3O)c2O)nc1C]

Examples of trianilino-s-triazine derivatives (d₁₃) which can be used according to the invention correspond to the formula

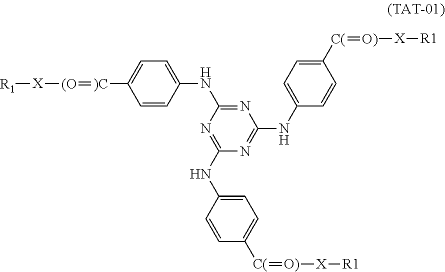

wherein
R₁, R₂ and R₃ independently of each other denote optionally substituted C₁-C₂₀-alkyl, aryl or hetaryl;
X denotes O; or NR₄; and
R₄ denotes hydrogen; or optionally substituted C₁-C₂₀-alkyl, aryl or hetaryl.

A particularly preferred representative of this compound class is Ethylhexyl Triazone corresponding to the formula

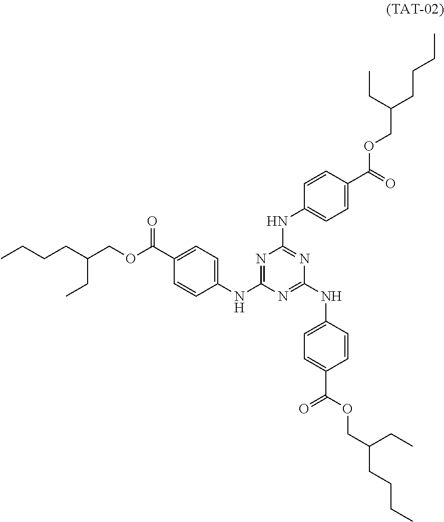 [SMILES: CCCCC(CC)COC(=O)c1ccc(Nc2nc(Nc3ccc(C(=O)OCC(CC)CCCC)cc3)nc(Nc3ccc(C(=O)OCC(CC)CCCC)cc3)n2)cc1]

or Diethylhexyl Butamido Triazone corresponding to the formula

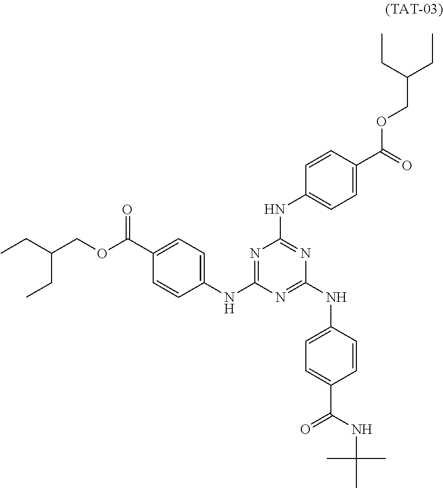 [SMILES: CCC(CC)COC(=O)c1ccc(Nc2nc(Nc3ccc(C(=O)NC(C)(C)C)cc3)nc(Nc3ccc(C(=O)OCC(CC)CC)cc3)n2)cc1]

or Ethylhexyl Bis-Isopentylbenzoxazolylphenyl Melamine corresponding to the formula

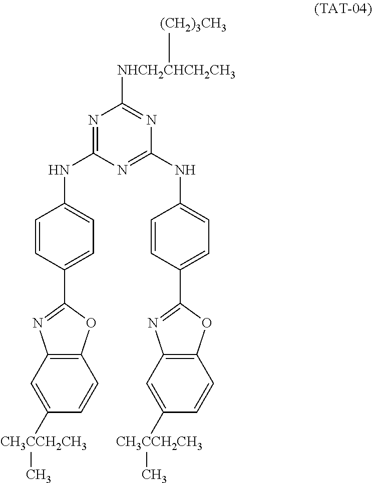

Preferred tris-biphenyl-triazine derivatives (d₁₇) which can be used according to the invention correspond to the formula

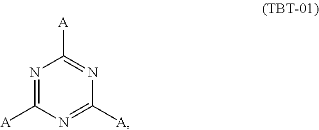

wherein
A denotes a radical of the formula

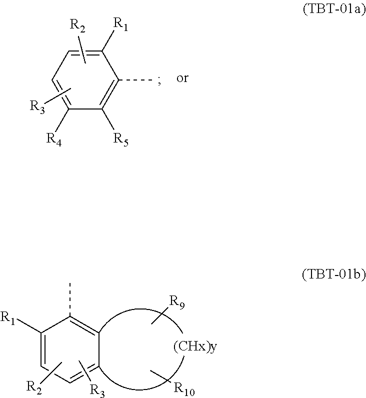

R₁ and R₅ independently of each other denote hydrogen; C₁-C₁₈-alkyl; or C₆-C₁₂-aryl;
R₂, R₃ and R₄ independently of each other denote hydrogen; or a radical of the formula (TBT-01c)

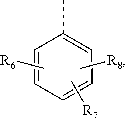

wherein in formula (TBT-01a) at least one of the radicals R₂, R₃ and R₄ denotes a radical of the formula (TBT-01c);
R₆, R₇, R₈, R₉ and R₁₀ independently of each other denote hydrogen; hydroxyl; halogen; C₁-C₁₈-alkyl; C₁-C₁₈-alkoxy; C₆-C₁₂-aryl; biphenylyl; C₆-C₁₂-aryloxy; C₁-C₁₈-alkylthio; carboxyl; —COOM; C₁-C₁₈-alkylcarboxyl; aminocarbonyl; or mono- or di-C₁-C₁₃-alkylamino; C₁-C₁₀-acylamino; —COOH;
M denotes an alkali metal ion;
x denotes 1 or 2; and
y denotes an integer from 2 to 10.

Preferably, the UV filters (d₁₇) which can be used according to the invention correspond to the compounds of the formula

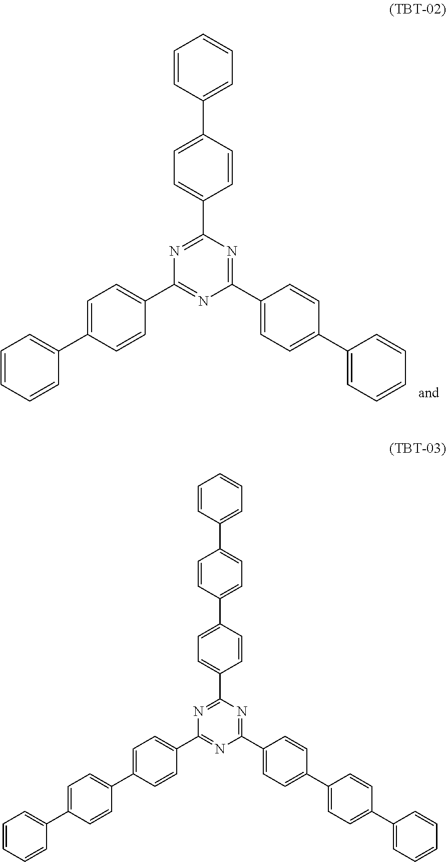 [SMILES: c1ccc(-c2ccc(-c3nc(-c4ccc(-c5ccccc5)cc4)nc(-c4ccc(-c5ccccc5)cc4)n3)cc2)cc1 ; c1ccc(-c2ccc(-c3ccc(-c4nc(-c5ccc(-c6ccc(-c7ccccc7)cc6)cc5)nc(-c5ccc(-c6ccc(-c7ccccc7)cc6)cc5)n4)cc3)cc2)cc1]

Preferred benzylidenemalonates (d₁₉) which can be employed according to the invention correspond to the formula

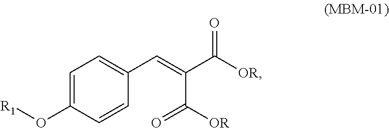

wherein
R₁ denotes methyl; ethyl; propyl; or n-butyl
if R₁ denotes methyl,
R denotes tert butyl;

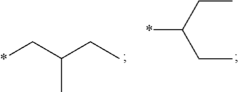

a radical of the formula

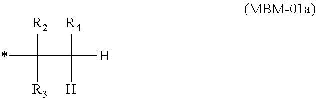

or a radical of the formula

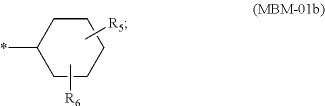

wherein
R₂ and R₃ independently of each other hydrogen; or methyl;
R₄ methyl; ethyl; or n-propyl;
R₅ and R₆ independently of each other hydrogen; or C₁-C₃-alkyl;
if R₁ denotes ethyl; propyl; or n-butyl,
R denotes isopropyl.

Particularly preferred benzylidenemalonates (d₁₉) which can be employed according to the invention are listed in the following table:

TABLE 1
Examples of benzylidenemalonates which can be used according to
the invention

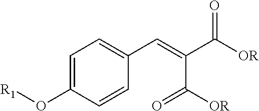

| | R₁ | R₂ |
|---|---|---|
| (MBM-02) | methyl | 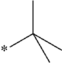 [SMILES: *C(C)(C)C] |
| (MBM-03) | methyl | 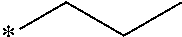 [SMILES: *CCC] |
| (MBM-04) | methyl | 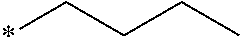 [SMILES: *CCCC] |
| (MBM-05) | methyl | 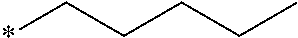 [SMILES: *CCCCC] |
| (MBM-06) | methyl | 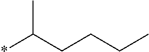 [SMILES: *C(C)CCCC] |
| (MBM-07) | methyl | 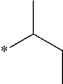 [SMILES: *C(C)CC] |
| (MBM-08) | methyl | 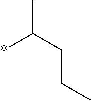 [SMILES: *C(C)CCC] |
| (MBM-09) | methyl | 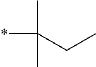 [SMILES: *C(C)(C)CC] |
| (MBM-10) | methyl | 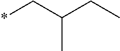 [SMILES: *CC(C)CC] |
| (MBM-11) | ethyl | 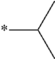 [SMILES: *C(C)C] |
| (MBM-12) | propyl | 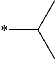 [SMILES: *C(C)C] |
| (MBM-13) | n-butyl | 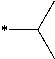 [SMILES: *C(C)C] |
| (MBM-14) | methyl | 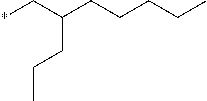 [SMILES: *CC(CCC)CCCCC] |
| (MBM-15) | methyl | 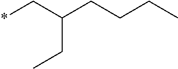 [SMILES: *CC(CC)CCCC] |

An example of a representative of phenylene-bis-diphenyltriazines (d₂₁) is 5,6,5,6-tetraphenyl-3,3′-(1,4-phenylene)-bis[1,2,4]triazine corresponds to the formula

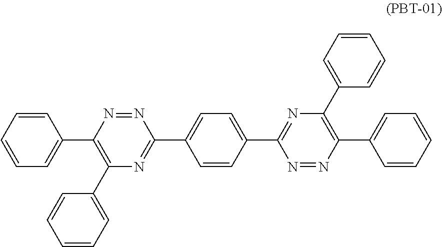 [SMILES: c1ccc(-c2nnc(-c3ccc(-c4nnc(-c5ccccc5)c(-c5ccccc5)n4)cc3)nc2-c2ccccc2)cc1]

An example of a representative of imidazoline derivatives is Ethyl hexyldimethoxybenzylidenedioxoimidazoline Propionate

An example of a representative of diarylbutadiene derivatives (d₂₃) is 1,1-dicarboxy-(2,2′-dimethylpropyl)-4,4-diphenylbutadiene.

Each of the abovementioned UV filters (d₁)-(d₂₃) can be used according to the invention as a mixture. For example, mixtures of two, three, four, five or six of the filter groups (d₁)-(d₂₃) can be used according to the invention. Mixtures of two, three, four, five or six UV filters from one or more representatives of substance classes (d₁)-(d₂₃) can also be used according to the invention.

Preferably, UV filters (d) are used according to the invention, in particular representatives of the following compound classes:
(d₁) p-aminobenzoic acid derivatives,
(d₂) salicylic acid derivatives,
(d₃) benzophenone derivatives,
(d₄) dibenzoylmethane derivatives,
(d₅) diphenyl acrylates,
(d₆) 3-imidazol-4-yl-acrylic acid and its esters;
(d₇) benzofuran derivatives;
(d₉) cinnamic acid derivatives;
(d₁₀) camphor derivatives;
(d₁₁) hydroxyphenyltriazine derivatives;
(d₁₂) benzotriazole derivatives;
(d₁₃) trianilino-s-triazine derivatives;
(d₁₅) menthyl o-aminobenzoates;
(d₁₆) homosalates;
(d₁₉) benzylidenemalonates; and
(d₂₀) merocyanine derivatives.

Particularly preferably, the following oil-soluble UV filters are used according to the invention:
(d_{SOL-1}) Benzophenone-3 (BP3);
(d_{SOL-2}) Benzophenone-4 (BP4);
(d_{SOL-3}) 3-Benzylidene Camphor (3BC);
(d_{SOL-4}) Bis-Ethylhexyloxyphenol Methoxyphenyl Triazine (BEMT);
(d_{SOL-5}) Butyl Methoxydibenzoylmethane (BMBM);
(d_{SOL-6}) Diethyl hexyl Butamido Triazone (DBT);
(d_{SOL-7}) Drometrizole Trisiloxane (DTS);
(d_{SOL-8}) Ethylhexyl Triazone (EHT);
(d_{SOL-9}) Ethylhexyl Methoxycinnamate;
(d_{SOL-10})Benzylidenemalonate (BM);
(d_{SOL-11}) Diethylamino Hydroxy Benzoyl Hexyl Benzoate (DHHB);
(d_{SOL-12}) Octocrylene;
(d_{SOL-13}) Polysilicone1-15;
(d_{SOL-14}) Homosalate; and
(d_{SOL-15}) Ethylhexyl salicylate.

UV filters which are very particularly preferred in the context of the present invention are oil-soluble filter mixtures of
(d₉ₐ) Ethylhexyl Methoxycinnamate,
(d₁₁ₐ) Bis-Ethylhexyloxyphenol Methoxyphenyl Triazine,
(d₁₃ₐ) Ethylhexyl Triazone and
(d₃ₐ) Diethylamino Hydroxy Benzoyl Hexyl Benzoate.

Possible UV filter mixtures according to the invention of oil-soluble UV filters (UV SOL 1-UV SOL 551) are listed in the table below:

TABLE 2
List of oil-soluble organic UV filter combinations

| | | | | | | | | | | | | |
|---|---|---|---|---|---|---|---|---|---|---|---|---|
| Abbreviation | BP3 | BP4 | 3BC | BEMT | BMBM | DBT | DTS | EHT | MBC | EMC | CD | DHHB |

1-Filter combinations

| | | | | | | | | | | | | |
|---|---|---|---|---|---|---|---|---|---|---|---|---|
| UV SOL 1 | x | | | | | | | | | | | |
| UV SOL 2 | | x | | | | | | | | | | |
| UV SOL 3 | | | x | | | | | | | | | |
| UV SOL 4 | | | | x | | | | | | | | |
| UV SOL 5 | | | | | x | | | | | | | |
| UV SOL 6 | | | | | | x | | | | | | |
| UV SOL 7 | | | | | | | x | | | | | |
| UV SOL 8 | | | | | | | | x | | | | |
| UV SOL 9 | | | | | | | | | x | | | |
| UV SOL 10 | | | | | | | | | | x | | |
| UV SOL 11 | | | | | | | | | | | x | |
| UV SOL 12 | | | | | | | | | | | | x |

2-Filter combinations

| | | | | | | | | | | | | |
|---|---|---|---|---|---|---|---|---|---|---|---|---|
| UV SOL 13 | x | x | | | | | | | | | | |
| UV SOL 14 | x | | x | | | | | | | | | |
| UV SOL 15 | x | | | x | | | | | | | | |
| UV SOL 16 | x | | | | x | | | | | | | |
| UV SOL 17 | x | | | | | x | | | | | | |
| UV SOL 18 | x | | | | | | x | | | | | |
| UV SOL 19 | x | | | | | | | x | | | | |
| UV SOL 20 | x | | | | | | | | x | | | |
| UV SOL 21 | x | | | | | | | | | x | | |
| UV SOL 22 | x | | | | | | | | | | x | |
| UV SOL 23 | x | | | | | | | | | | | x |
| UV SOL 24 | | x | x | | | | | | | | | |
| UV SOL 25 | | x | | x | | | | | | | | |
| UV SOL 26 | | x | | | x | | | | | | | |
| UV SOL 27 | | x | | | | x | | | | | | |
| UV SOL 28 | | x | | | | | x | | | | | |
| UV SOL 29 | | x | | | | | | x | | | | |
| UV SOL 30 | | x | | | | | | | x | | | |
| UV SOL 31 | | x | | | | | | | | x | | |
| UV SOL 32 | | x | | | | | | | | | x | |
| UV SOL 33 | | x | | | | | | | | | | x |
| UV SOL 34 | | | x | x | | | | | | | | |
| UV SOL 35 | | | x | | x | | | | | | | |
| UV SOL 36 | | | x | | | x | | | | | | |
| UV SOL 37 | | | x | | | | x | | | | | |
| UV SOL 38 | | | x | | | | | x | | | | |
| UV SOL 39 | | | x | | | | | | x | | | |
| UV SOL 40 | | | x | | | | | | | x | | |
| UV SOL 41 | | | x | | | | | | | | x | |
| UV SOL 42 | | | x | | | | | | | | | x |
| UV SOL 43 | | | | x | x | | | | | | | |
| UV SOL 44 | | | | x | | x | | | | | | |
| UV SOL 45 | | | | x | | | x | | | | | |
| UV SOL 46 | | | | x | | | | x | | | | |
| UV SOL 47 | | | | x | | | | | x | | | |
| UV SOL 48 | | | | x | | | | | | x | | |
| UV SOL 49 | | | | x | | | | | | | x | |
| UV SOL 50 | | | | x | | | | | | | | x |
| UV SOL 51 | | | | | x | x | | | | | | |
| UV SOL 52 | | | | | x | | x | | | | | |
| UV SOL 53 | | | | | x | | | x | | | | |
| UV SOL 54 | | | | | x | | | | x | | | |
| UV SOL 55 | | | | | x | | | | | x | | |
| UV SOL 56 | | | | | x | | | | | | x | |
| UV SOL 57 | | | | | x | | | | | | | x |
| UV SOL 58 | | | | | | x | x | | | | | |
| UV SOL 59 | | | | | | x | | x | | | | |
| UV SOL 60 | | | | | | x | | | x | | | |
| UV SOL 61 | | | | | | x | | | | x | | |
| UV SOL 62 | | | | | | x | | | | | x | |
| UV SOL 63 | | | | | | x | | | | | | x |
| UV SOL 64 | | | | | | | x | x | | | | |
| UV SOL 65 | | | | | | | x | | x | | | |
| UV SOL 66 | | | | | | | x | | | x | | |
| UV SOL 67 | | | | | | | x | | | | x | |
| UV SOL 68 | | | | | | | x | | | | | x |
| UV SOL 69 | | | | | | | | x | x | | | |
| UV SOL 70 | | | | | | | | x | | x | | |
| UV SOL 71 | | | | | | | | x | | | x | |
| UV SOL 72 | | | | | | | | x | | | | x |
| UV SOL 73 | | | | | | | | | x | x | | |
| UV SOL 74 | | | | | | | | | x | | x | |
| UV SOL 75 | | | | | | | | | x | | | x |
| UV SOL 76 | | | | | | | | | | x | x | |
| UV SOL 77 | | | | | | | | | | x | | x |
| UV SOL 78 | | | | | | | | | | | x | x |

3-Filter combinations

| | | | | | | | | | | | | |
|---|---|---|---|---|---|---|---|---|---|---|---|---|
| UV SOL 79 | x | x | x | | | | | | | | | |
| UV SOL 80 | x | x | | x | | | | | | | | |
| UV SOL 81 | x | x | | | x | | | | | | | |
| UV SOL 82 | x | x | | | | x | | | | | | |
| UV SOL 83 | x | x | | | | | x | | | | | |
| UV SOL 84 | x | x | | | | | | x | | | | |
| UV SOL 86 | x | x | | | | | | | x | | | |
| UV SOL 87 | x | x | | | | | | | | x | | |
| UV SOL 88 | x | x | | | | | | | | | x | |
| UV SOL 89 | x | x | | | | | | | | | | x |
| UV SOL 90 | x | | x | x | | | | | | | | |
| UV SOL 91 | x | | x | | x | | | | | | | |
| UV SOL 92 | x | | x | | | x | | | | | | |
| UV SOL 93 | x | | x | | | | x | | | | | |
| UV SOL 94 | x | | x | | | | | x | | | | |
| UV SOL 95 | x | | x | | | | | | x | | | |
| UV SOL 96 | x | | x | | | | | | | x | | |
| UV SOL 97 | x | | x | | | | | | | | x | |
| UV SOL 98 | x | | x | | | | | | | | | x |
| UV SOL 99 | x | | | x | x | | | | | | | |
| UV SOL 100 | x | | | x | | x | | | | | | |
| UV SOL 101 | x | | | x | | | x | | | | | |
| UV SOL 102 | x | | | x | | | | x | | | | |
| UV SOL 103 | x | | | x | | | | | x | | | |
| UV SOL 104 | x | | | x | | | | | | x | | |
| UV SOL 105 | x | | | x | | | | | | | x | |
| UV SOL 106 | x | | | x | | | | | | | | x |
| UV SOL 107 | x | | | | x | x | | | | | | |
| UV SOL 108 | x | | | | x | | x | | | | | |
| UV SOL 109 | x | | | | x | | | x | | | | |
| UV SOL 110 | x | | | | x | | | | x | | | |
| UV SOL 111 | x | | | | x | | | | | x | | |
| UV SOL 112 | x | | | | x | | | | | | x | |
| UV SOL 113 | x | | | | x | | | | | | | x |
| UV SOL 114 | x | | | | | x | x | | | | | |
| UV SOL 115 | x | | | | | x | | x | | | | |
| UV SOL 116 | x | | | | | x | | | x | | | |
| UV SOL 117 | x | | | | | x | | | | x | | |
| UV SOL 118 | x | | | | | x | | | | | x | |
| UV SOL 119 | x | | | | | x | | | | | | x |
| UV SOL 120 | x | | | | | | x | x | | | | |
| UV SOL 121 | x | | | | | | x | | x | | | |
| UV SOL 122 | x | | | | | | x | | | x | | |
| UV SOL 123 | x | | | | | | x | | | | x | |
| UV SOL 124 | x | | | | | | x | | | | | x |
| UV SOL 125 | x | | | | | | | x | x | | | |
| UV SOL 126 | x | | | | | | | x | | x | | |
| UV SOL 127 | x | | | | | | | x | | | x | |
| UV SOL 128 | x | | | | | | | x | | | | x |
| UV SOL 129 | x | | | | | | | | x | x | | |
| UV SOL 130 | x | | | | | | | | x | | x | |
| UV SOL 131 | x | | | | | | | | x | | | x |
| UV SOL 132 | x | | | | | | | | | x | x | |
| UV SOL 133 | x | | | | | | | | | x | | x |
| UV SOL 134 | x | | | | | | | | | | x | x |
| UV SOL 135 | | x | x | x | | | | | | | | |
| UV SOL 136 | | x | x | | x | | | | | | | |
| UV SOL 137 | | x | x | | | x | | | | | | |
| UV SOL 138 | | x | x | | | | x | | | | | |
| UV SOL 139 | | x | x | | | | | x | | | | |
| UV SOL 140 | | x | x | | | | | | x | | | |
| UV SOL 141 | | x | x | | | | | | | x | | |
| UV SOL 142 | | x | x | | | | | | | | x | |
| UV SOL 143 | | x | x | | | | | | | | | x |
| UV SOL 144 | | x | | x | x | | | | | | | |
| UV SOL 145 | | x | | x | | x | | | | | | |
| UV SOL 146 | | x | | x | | | x | | | | | |
| UV SOL 147 | | x | | x | | | | x | | | | |
| UV SOL 148 | | x | | x | | | | | x | | | |
| UV SOL 149 | | x | | x | | | | | | x | | |
| UV SOL 150 | | x | | x | | | | | | | x | |
| UV SOL 151 | | x | | x | | | | | | | | x |
| UV SOL 152 | | x | | | x | x | | | | | | |
| UV SOL 153 | | x | | | x | | x | | | | | |
| UV SOL 154 | | x | | | x | | | x | | | | |
| UV SOL 155 | | x | | | x | | | | x | | | |
| UV SOL 156 | | x | | | x | | | | | x | | |
| UV SOL 157 | | x | | | x | | | | | | x | |
| UV SOL 158 | | x | | | x | | | | | | | x |
| UV SOL 159 | | x | | | | x | x | | | | | |
| UV SOL 160 | | x | | | | x | | x | | | | |
| UV SOL 161 | | x | | | | x | | | x | | | |
| UV SOL 162 | | x | | | | x | | | | x | | |
| UV SOL 163 | | x | | | | x | | | | | x | |
| UV SOL 164 | | x | | | | x | | | | | | x |
| UV SOL 165 | | x | | | | | x | x | | | | |
| UV SOL 166 | | x | | | | | x | | x | | | |
| UV SOL 167 | | x | | | | | x | | | x | | |
| UV SOL 168 | | x | | | | | x | | | | x | |
| UV SOL 169 | | x | | | | | x | | | | | x |
| UV SOL 170 | | x | | | | | | x | x | | | |
| UV SOL 171 | | x | | | | | | x | | x | | |
| UV SOL 172 | | x | | | | | | x | | | x | |
| UV SOL 173 | | x | | | | | | x | | | | x |
| UV SOL 174 | | x | | | | | | | x | x | | |
| UV SOL 175 | | x | | | | | | | x | | x | |
| UV SOL 176 | | x | | | | | | | x | | | x |
| UV SOL 177 | | x | | | | | | | | x | x | |
| UV SOL 178 | | x | | | | | | | | x | | x |
| UV SOL 179 | | x | | | | | | | | | x | x |
| UV SOL 180 | | | x | x | x | | | | | | | |
| UV SOL 181 | | | x | x | | x | | | | | | |
| UV SOL 182 | | | x | x | | | x | | | | | |
| UV SOL 183 | | | x | x | | | | x | | | | |
| UV SOL 184 | | | x | x | | | | | x | | | |
| UV SOL 185 | | | x | x | | | | | | x | | |
| UV SOL 186 | | | x | x | | | | | | | x | |
| UV SOL 187 | | | x | x | | | | | | | | x |
| UV SOL 188 | | | x | | x | x | | | | | | |
| UV SOL 189 | | | x | | x | | x | | | | | |
| UV SOL 190 | | | x | | x | | | x | | | | |
| UV SOL 191 | | | x | | x | | | | x | | | |
| UV SOL 192 | | | x | | x | | | | | x | | |
| UV SOL 193 | | | x | | x | | | | | | x | |
| UV SOL 194 | | | x | | x | | | | | | | x |
| UV SOL 195 | | | x | | | x | x | | | | | |
| UV SOL 196 | | | x | | | x | | x | | | | |
| UV SOL 197 | | | x | | | x | | | x | | | |
| UV SOL 198 | | | x | | | x | | | | x | | |
| UV SOL 199 | | | x | | | x | | | | | x | |
| UV SOL 200 | | | x | | | x | | | | | | x |
| UV SOL 201 | | | x | | | | x | x | | | | |
| UV SOL 202 | | | x | | | | x | | x | | | |
| UV SOL 203 | | | x | | | | x | | | x | | |
| UV SOL 204 | | | x | | | | x | | | | x | |
| UV SOL 205 | | | x | | | | x | | | | | x |
| UV SOL 206 | | | x | | | | | x | x | | | |
| UV SOL 207 | | | x | | | | | x | | x | | |
| UV SOL 208 | | | x | | | | | x | | | x | |
| UV SOL 209 | | | x | | | | | x | | | | x |
| UV SOL 210 | | | x | | | | | | x | x | | |
| UV SOL 211 | | | x | | | | | | x | | x | |
| UV SOL 212 | | | x | | | | | | x | | | x |
| UV SOL 213 | | | x | | | | | | | x | x | |
| UV SOL 214 | | | x | | | | | | | x | | x |
| UV SOL 215 | | | x | | | | | | | | x | x |
| UV SOL 216 | | | | x | x | x | | | | | | |
| UV SOL 217 | | | | x | x | | x | | | | | |
| UV SOL 218 | | | | x | x | | | x | | | | |
| UV SOL 219 | | | | x | x | | | | x | | | |
| UV SOL 220 | | | | x | x | | | | | x | | |
| UV SOL 221 | | | | x | x | | | | | | x | |
| UV SOL 222 | | | | x | x | | | | | | | x |
| UV SOL 223 | | | | x | | x | x | | | | | |
| UV SOL 224 | | | | x | | x | | x | | | | |
| UV SOL 225 | | | | x | | x | | | x | | | |
| UV SOL 226 | | | | x | | x | | | | x | | |
| UV SOL 227 | | | | x | | x | | | | | x | |
| UV SOL 228 | | | | x | | x | | | | | | x |
| UV SOL 229 | | | | x | | | x | x | | | | |
| UV SOL 230 | | | | x | | | x | | x | | | |
| UV SOL 231 | | | | x | | | x | | | x | | |
| UV SOL 232 | | | | x | | | x | | | | x | |
| UV SOL 233 | | | | x | | | x | | | | | x |
| UV SOL 234 | | | | x | | | | x | x | | | |
| UV SOL 235 | | | | x | | | | x | | x | | |
| UV SOL 236 | | | | x | | | | x | | | x | |
| UV SOL 237 | | | | x | | | | x | | | | x |
| UV SOL 238 | | | | x | | | | | x | x | | |
| UV SOL 239 | | | | x | | | | | x | | x | |
| UV SOL 240 | | | | x | | | | | x | | | x |
| UV SOL 241 | | | | x | | | | | | x | x | |
| UV SOL 242 | | | | x | | | | | | x | | x |
| UV SOL 243 | | | | x | | | | | | | x | x |
| UV SOL 244 | | | | | x | x | x | | | | | |
| UV SOL 245 | | | | | x | x | | x | | | | |
| UV SOL 246 | | | | | x | x | | | x | | | |
| UV SOL 247 | | | | | x | x | | | | x | | |
| UV SOL 248 | | | | | x | x | | | | | x | |
| UV SOL 249 | | | | | x | x | | | | | | x |
| UV SOL 250 | | | | | x | | x | x | | | | |
| UV SOL 251 | | | | | x | | x | | x | | | |
| UV SOL 252 | | | | | x | | x | | | x | | |
| UV SOL 253 | | | | | x | | x | | | | x | |
| UV SOL 254 | | | | | x | | x | | | | | x |
| UV SOL 255 | | | | | x | | | x | x | | | |
| UV SOL 256 | | | | | x | | | x | | x | | |
| UV SOL 257 | | | | | x | | | x | | | x | |
| UV SOL 258 | | | | | x | | | x | | | | x |
| UV SOL 259 | | | | | x | | | | x | x | | |
| UV SOL 260 | | | | | x | | | | x | | x | |
| UV SOL 261 | | | | | x | | | | x | | | x |
| UV SOL 262 | | | | | x | | | | | x | x | |
| UV SOL 263 | | | | | x | | | | | x | | x |
| UV SOL 264 | | | | | x | | | | | | x | x |
| UV SOL 265 | | | | | | x | x | x | | | | |
| UV SOL 266 | | | | | | x | x | | x | | | |
| UV SOL 267 | | | | | | x | x | | | x | | |
| UV SOL 268 | | | | | | x | x | | | | x | |
| UV SOL 289 | | | | | | x | x | | | | | x |
| UV SOL 290 | | | | | | x | | x | x | | | |
| UV SOL 291 | | | | | | x | | x | | x | | |
| UV SOL 292 | | | | | | x | | x | | | x | |
| UV SOL 293 | | | | | | x | | x | | | | x |
| UV SOL 294 | | | | | | x | | | x | x | | |
| UV SOL 295 | | | | | | x | | | x | | x | |
| UV SOL 296 | | | | | | x | | | x | | | x |
| UV SOL 297 | | | | | | x | | | | x | x | |
| UV SOL 298 | | | | | | x | | | | x | | x |
| UV SOL 299 | | | | | | x | | | | | x | x |
| UV SOL 300 | | | | | | | x | x | x | | | |
| UV SOL 301 | | | | | | | x | x | | x | | |
| UV SOL 302 | | | | | | | x | x | | | x | |
| UV SOL 303 | | | | | | | x | x | | | | x |
| UV SOL 304 | | | | | | | x | | x | x | | |
| UV SOL 305 | | | | | | | x | | x | | x | |
| UV SOL 306 | | | | | | | x | | x | | | x |
| UV SOL 307 | | | | | | | x | | | x | x | |
| UV SOL 308 | | | | | | | x | | | x | | X |
| UV SOL 309 | | | | | | | x | | | | x | x |
| UV SOL 310 | | | | | | | | x | x | x | | |
| UV SOL 311 | | | | | | | | x | x | | x | |
| UV SOL 312 | | | | | | | | x | x | | | x |
| UV SOL 313 | | | | | | | | x | | x | x | |
| UV SOL 314 | | | | | | | | x | | x | | x |
| UV SOL 315 | | | | | | | | x | | | x | x |
| UV SOL 316 | | | | | | | | | x | x | x | |
| UV SOL 317 | | | | | | | | | x | x | | x |
| UV SOL 318 | | | | | | | | | x | | x | x |
| UV SOL 319 | | | | | | | | | | x | x | x |

4-Filter combinations

| | | | | | | | | | | | | |
|---|---|---|---|---|---|---|---|---|---|---|---|---|
| UV SOL 320 | x | x | x | x | | | | | | | | |
| UV SOL 321 | x | x | x | | x | | | | | | | |
| UV SOL 322 | x | x | x | | | x | | | | | | |
| UV SOL 323 | x | x | x | | | | x | | | | | |
| UV SOL 324 | x | x | x | | | | | x | | | | |
| UV SOL 325 | x | x | x | | | | | | x | | | |
| UV SOL 326 | x | x | x | | | | | | | x | | |
| UV SOL 327 | x | x | x | | | | | | | | x | |
| UV SOL 328 | x | x | x | | | | | | | | | x |
| UV SOL 329 | x | x | | x | x | | | | | | | |
| UV SOL 330 | x | x | | x | | x | | | | | | |
| UV SOL 331 | x | x | | x | | | x | | | | | |
| UV SOL 332 | x | x | | x | | | | x | | | | |
| UV SOL 333 | x | x | | x | | | | | x | | | |
| UV SOL 334 | x | x | | x | | | | | | x | | |
| UV SOL 335 | x | x | | x | | | | | | | X | |
| UV SOL 336 | x | x | | x | | | | | | | | x |
| UV SOL 337 | x | x | | | x | x | | | | | | |
| UV SOL 338 | x | x | | | x | | x | | | | | |
| UV SOL 339 | x | x | | | x | | | x | | | | |
| UV SOL 340 | x | x | | | x | | | | x | | | |
| UV SOL 341 | x | x | | | x | | | | | x | | |
| UV SOL 342 | x | x | | | x | | | | | | x | |
| UV SOL 343 | x | x | | | x | | | | | | | x |
| UV SOL 344 | x | x | | | | x | X | | | | | |
| UV SOL 345 | x | x | | | | x | | x | | | | |
| UV SOL 346 | x | x | | | | x | | | x | | | |
| UV SOL 347 | x | x | | | | x | | | | x | | |
| UV SOL 348 | x | x | | | | x | | | | | x | |
| UV SOL 349 | x | x | | | | x | | | | | | x |
| UV SOL 350 | x | x | | | | | x | x | | | | |
| UV SOL 351 | x | x | | | | | x | | x | | | |
| UV SOL 352 | x | x | | | | | x | | | x | | |
| UV SOL 353 | x | x | | | | | x | | | | x | |
| UV SOL 354 | x | x | | | | | x | | | | | x |
| UV SOL 355 | x | x | | | | | | x | x | | | |
| UV SOL 356 | x | x | | | | | | x | | x | | |
| UV SOL 357 | x | x | | | | | | x | | | x | |
| UV SOL 358 | x | x | | | | | | x | | | | x |
| UV SOL 359 | x | x | | | | | | | x | x | | |
| UV SOL 360 | x | x | | | | | | | x | | x | |
| UV SOL 361 | x | x | | | | | | | x | | | x |
| UV SOL 362 | x | x | | | | | | | | x | x | |
| UV SOL 363 | x | x | | | | | | | | x | | x |
| UV SOL 364 | x | x | | | | | | | | | x | x |
| UV SOL 365 | x | | x | x | x | | | | | | | |
| UV SOL 366 | x | | x | x | | x | | | | | | |
| UV SOL 367 | x | | x | x | | | x | | | | | |
| UV SOL 368 | x | | x | x | | | | x | | | | |
| UV SOL 369 | x | | x | x | | | | | x | | | |
| UV SOL 370 | x | | x | x | | | | | | x | | |
| UV SOL 371 | x | | x | x | | | | | | | x | |
| UV SOL 372 | x | | x | x | | | | | | | | x |
| UV SOL 373 | x | | x | | x | x | | | | | | |
| UV SOL 374 | x | | x | | x | | x | | | | | |
| UV SOL 375 | x | | x | | x | | | x | | | | |
| UV SOL 376 | x | | x | | x | | | | x | | | |
| UV SOL 377 | x | | x | | x | | | | | x | | |
| UV SOL 378 | x | | x | | x | | | | | | x | |
| UV SOL 379 | x | | x | | x | | | | | | | x |
| UV SOL 380 | x | | x | | | x | x | | | | | |
| UV SOL 381 | x | | x | | | x | | x | | | | |
| UV SOL 382 | x | | x | | | x | | | x | | | |
| UV SOL 383 | x | | x | | | x | | | | x | | |
| UV SOL 384 | x | | x | | | x | | | | | x | |
| UV SOL 385 | x | | x | | | x | | | | | | x |
| UV SOL 386 | x | | x | | | | x | x | | | | |
| UV SOL 387 | x | | x | | | | x | | x | | | |
| UV SOL 388 | x | | x | | | | x | | | x | | |
| UV SOL 389 | x | | x | | | | x | | | | x | |
| UV SOL 390 | x | | x | | | | x | | | | | x |
| UV SOL 391 | x | | x | | | | | x | x | | | |
| UV SOL 392 | x | | x | | | | | x | | x | | |
| UV SOL 393 | x | | x | | | | | x | | | x | |
| UV SOL 394 | x | | x | | | | | x | | | | x |
| UV SOL 395 | x | | x | | | | | | x | x | | |
| UV SOL 396 | x | | x | | | | | | x | | x | |
| UV SOL 397 | x | | x | | | | | | x | | | x |
| UV SOL 398 | x | | x | | | | | | | x | x | |
| UV SOL 399 | x | | x | | | | | | | x | | x |
| UV SOL 400 | x | | x | | | | | | | | x | x |
| UV SOL 401 | x | | | x | x | x | | | | | | |
| UV SOL 402 | x | | | x | x | | x | | | | | |
| UV SOL 403 | x | | | x | x | | | x | | | | |
| UV SOL 404 | x | | | x | x | | | | x | | | |
| UV SOL 405 | x | | | x | x | | | | | x | | |
| UV SOL 406 | x | | | x | x | | | | | | x | |
| UV SOL 407 | X | | | x | x | | | | | | | x |
| UV SOL 408 | x | | | x | | x | x | | | | | |
| UV SOL 409 | x | | | x | | x | | x | | | | |
| UV SOL 410 | x | | | x | | x | | | x | | | |
| UV SOL 411 | x | | | x | | x | | | | x | | |
| UV SOL 412 | x | | | x | | x | | | | | x | |
| UV SOL 413 | x | | | x | | x | | | | | | x |
| UV SOL 414 | x | | | x | | | x | x | | | | |
| UV SOL 415 | x | | | x | | | x | | x | | | |
| UV SOL 416 | x | | | x | | | x | | | x | | |
| UV SOL 417 | x | | | x | | | x | | | | x | |
| UV SOL 418 | x | | | x | | | x | | | | | x |
| UV SOL 419 | x | | | x | | | | x | x | | | |
| UV SOL 420 | x | | | x | | | | x | | x | | |
| UV SOL 421 | x | | | x | | | | x | | | x | |
| UV SOL 422 | x | | | x | | | | x | | | | x |
| UV SOL 423 | x | | | x | | | | | x | x | | |
| UV SOL 424 | x | | | x | | | | | x | | x | |
| UV SOL 425 | x | | | x | | | | | x | | | x |
| UV SOL 426 | x | | | x | | | | | | x | x | |
| UV SOL 427 | x | | | x | | | | | | x | | x |
| UV SOL 428 | x | | | x | | | | | | | x | x |
| UV SOL 429 | x | | | | x | x | x | | | | | |
| UV SOL 430 | x | | | | x | x | | x | | | | |
| UV SOL 431 | x | | | | x | x | | | x | | | |
| UV SOL 432 | x | | | | x | x | | | | x | | |
| UV SOL 433 | x | | | | x | x | | | | | x | |
| UV SOL 434 | x | | | | x | x | | | | | | x |
| UV SOL 435 | x | | | | x | | x | x | | | | |
| UV SOL 436 | x | | | | x | | x | | x | | | |
| UV SOL 437 | x | | | | x | | x | | | x | | |
| UV SOL 438 | x | | | | x | | x | | | | x | |
| UV SOL 439 | x | | | | x | | x | | | | | x |
| UV SOL 440 | x | | | | x | | | x | x | | | |
| UV SOL 441 | x | | | | x | | | x | | x | | |
| UV SOL 442 | x | | | | x | | | x | | | x | |
| UV SOL 443 | x | | | | x | | | x | | | | x |
| UV SOL 444 | x | | | | x | | | | x | x | | |
| UV SOL 445 | x | | | | x | | | | x | | x | |
| UV SOL 446 | x | | | | x | | | | x | | | x |
| UV SOL 447 | x | | | | x | | | | | x | x | |
| UV SOL 448 | x | | | | x | | | | | x | | x |
| UV SOL 449 | x | | | | x | | | | | | x | x |
| UV SOL 450 | x | | | | | x | x | x | | | | |
| UV SOL 451 | x | | | | | x | x | | x | | | |
| UV SOL 452 | x | | | | | x | x | | | x | | |
| UV SOL 453 | x | | | | | x | x | | | | x | |
| UV SOL 454 | x | | | | | x | x | | | | | x |
| UV SOL 455 | x | | | | | x | | x | x | | | |
| UV SOL 456 | x | | | | | x | | x | | x | | |
| UV SOL 457 | x | | | | | x | | x | | | x | |
| UV SOL 458 | x | | | | | x | | x | | | | x |
| UV SOL 459 | x | | | | | x | | | x | x | | |
| UV SOL 460 | x | | | | | x | | | x | | x | |
| UV SOL 461 | x | | | | | x | | | x | | | x |
| UV SOL 462 | x | | | | | x | | | | x | x | |
| UV SOL 463 | x | | | | | x | | | | x | | x |
| UV SOL 464 | x | | | | | x | | | | | x | x |
| UV SOL 465 | x | | | | | | x | x | x | | | |
| UV SOL 466 | x | | | | | | x | x | | x | | |
| UV SOL 467 | x | | | | | | x | x | | | x | |
| UV SOL 468 | x | | | | | | x | x | | | | x |
| UV SOL 469 | x | | | | | | x | | x | x | | |
| UV SOL 470 | x | | | | | | x | | x | | x | |
| UV SOL 471 | x | | | | | | x | | x | | | x |
| UV SOL 472 | x | | | | | | x | | | x | x | |
| UV SOL 473 | x | | | | | | x | | | x | | x |
| UV SOL 474 | x | | | | | | x | | | | x | x |
| UV SOL 475 | x | | | | | | | x | x | x | | |
| UV SOL 476 | x | | | | | | | x | x | | x | |
| UV SOL 477 | x | | | | | | | x | x | | | x |
| UV SOL 478 | x | | | | | | | x | | x | x | |
| UV SOL 479 | x | | | | | | | x | | x | | x |
| UV SOL 480 | x | | | | | | | x | | | x | x |
| UV SOL 481 | x | | | | | | | | x | x | x | |
| UV SOL 482 | x | | | | | | | | x | x | | x |
| UV SOL 483 | x | | | | | | | | x | | x | x |
| UV SOL 484 | x | | | | | | | | | x | x | x |
| UV SOL 485 | | x | x | x | x | | | | | | | |
| UV SOL 486 | | x | x | x | x | x | | | | | | |
| UV SOL 487 | | x | x | x | x | | x | | | | | |
| UV SOL 488 | | x | x | x | x | | | x | | | | |
| UV SOL 489 | | x | x | x | x | | | | x | | | |
| UV SOL 490 | | x | x | x | x | | | | | x | | |
| UV SOL 491 | | x | x | x | x | | | | | | x | |
| UV SOL 492 | | x | x | x | x | | | | | | | x |
| UV SOL 493 | | x | x | | x | x | | | | | | |
| UV SOL 494 | | x | x | | x | | x | | | | | |
| UV SOL 495 | | x | x | | x | | | x | | | | |
| UV SOL 496 | | x | x | | x | | | | x | | | |
| UV SOL 497 | | x | x | | x | | | | | x | | |
| UV SOL 498 | | x | x | | x | | | | | | x | |
| UV SOL 499 | | x | x | | x | | | | | | | x |
| UV SOL 490 | | x | x | | | x | x | | | | | |
| UV SOL 491 | | x | x | | | x | | x | | | | |
| UV SOL 492 | | x | x | | | x | | | x | | | |
| UV SOL 493 | | x | x | | | x | | | | x | | |
| UV SOL 494 | | x | x | | | x | | | | | x | |
| UV SOL 495 | | x | x | | | x | | | | | | x |
| UV SOL 496 | | x | x | | | | x | x | | | | |
| UV SOL 497 | | x | x | | | | x | | x | | | |
| UV SOL 498 | | x | x | | | | x | | | x | | |
| UV SOL 499 | | x | x | | | | x | | | | x | |
| UV SOL 500 | | x | x | | | | x | | | | | x |
| UV SOL 501 | | x | x | | | | | x | x | | | |
| UV SOL 502 | | x | x | | | | | x | | x | | |
| UV SOL 503 | | x | x | | | | | x | | | x | |
| UV SOL 504 | | x | x | | | | | x | | | | x |
| UV SOL 505 | | x | x | | | | | | x | x | | |
| UV SOL 506 | | x | x | | | | | | x | | x | |
| UV SOL 507 | | x | x | | | | | | x | | | x |
| UV SOL 508 | | x | x | | | | | | | x | x | |
| UV SOL 509 | | x | x | | | | | | | x | | x |
| UV SOL 510 | | x | x | | | | | | | | x | x |
| UV SOL 511 | | | x | x | x | x | | | | | | |
| UV SOL 512 | | | x | x | x | | x | | | | | |
| UV SOL 513 | | | x | x | x | | | x | | | | |
| UV SOL 514 | | | x | x | x | | | | x | | | |
| UV SOL 515 | | | x | x | x | | | | | x | | |
| UV SOL 516 | | | x | x | x | | | | | | x | |
| UV SOL 517 | | | x | x | x | | | | | | | x |
| UV SOL 518 | | | x | x | | x | x | | | | | |
| UV SOL 519 | | | x | x | | x | | x | | | | |
| UV SOL 520 | | | x | x | | x | | | x | | | |
| UV SOL 521 | | | x | x | | x | | | | x | | |
| UV SOL 522 | | | x | x | | x | | | | | x | |
| UV SOL 523 | | | x | x | | x | | | | | | x |
| UV SOL 524 | | | x | x | | | x | x | | | | |
| UV SOL 525 | | | x | x | | | x | | x | | | |
| UV SOL 526 | | | x | x | | | x | | | x | | |
| UV SOL 527 | | | x | x | | | x | | | | x | |
| UV SOL 528 | | | x | x | | | x | | | | | x |
| UV SOL 529 | | | x | x | | | | x | x | | | |
| UV SOL 530 | | | x | x | | | | x | | x | | |
| UV SOL 531 | | | x | x | | | | x | | | x | |
| UV SOL 532 | | | x | x | | | | x | | | | x |
| UV SOL 533 | | | x | x | | | | | x | x | | |
| UV SOL 534 | | | x | x | | | | | x | | x | |
| UV SOL 535 | | | x | x | | | | | x | | | x |
| UV SOL 536 | | | x | x | | | | | | x | x | |
| UV SOL 537 | | | x | x | | | | | | x | | x |
| UV SOL 538 | | | x | x | | | | | | | x | x |
| UV SOL 539 | | | | x | x | x | x | | | | | |
| UV SOL 540 | | | | x | x | x | | x | | | | |
| UV SOL 541 | | | | x | x | x | | | x | | | |
| UV SOL 542 | | | | x | x | x | | | | x | | |
| UV SOL 543 | | | | x | x | x | | | | | x | |
| UV SOL 544 | | | | x | x | x | | | | | | x |
| UV SOL 545 | | | | x | x | | x | x | | | | |
| UV SOL 546 | | | | x | x | | x | | x | | | |
| UV SOL 547 | | | | x | x | | x | | | x | | |
| UV SOL 548 | | | | x | x | | x | | | | x | |
| UV SOL 549 | | | | x | x | | x | | | | | x |
| UV SOL 550 | | | | x | x | | | x | x | | | |
| UV SOL 551 | | | | x | x | | | x | | x | | |
| UV SOL 552 | | | | x | x | | | x | | | x | |
| UV SOL 553 | | | | x | x | | | x | | | | x |
| UV SOL 554 | | | | x | x | | | | x | x | | |
| UV SOL 555 | | | | x | x | | | | x | | x | |
| UV SOL 556 | | | | x | x | | | | x | | | x |
| UV SOL 557 | | | | x | x | | | | | x | x | |
| UV SOL 558 | | | | x | x | | | | | x | | x |
| UV SOL 559 | | | | x | x | | | | | | x | x |
| UV SOL 560 | | | | | x | x | x | x | | | | |
| UV SOL 561 | | | | | x | x | x | | x | | | |
| UV SOL 562 | | | | | x | x | x | | | x | | |
| UV SOL 563 | | | | | x | x | x | | | | x | |
| UV SOL 564 | | | | | x | x | x | | | | | x |
| UV SOL 565 | | | | | x | x | | x | x | | | |
| UV SOL 566 | | | | | x | x | | x | | x | | |
| UV SOL 567 | | | | | x | x | | x | | | x | |
| UV SOL 568 | | | | | x | x | | x | | | | x |
| UV SOL 569 | | | | | x | x | | | x | x | | |
| UV SOL 570 | | | | | x | x | | | x | | x | |
| UV SOL 571 | | | | | x | x | | | x | | | x |
| UV SOL 572 | | | | | x | x | | | | x | x | |
| UV SOL 573 | | | | | x | x | | | | x | | x |
| UV SOL 574 | | | | | x | x | | | | | x | x |
| UV SOL 575 | | | | | | x | x | x | x | | | |
| UV SOL 576 | | | | | | x | x | x | | x | | |
| UV SOL 577 | | | | | | x | x | x | | | x | |
| UV SOL 578 | | | | | | x | x | x | | | | x |
| UV SOL 579 | | | | | | x | x | | x | x | | |
| UV SOL 580 | | | | | | x | x | | x | | x | |
| UV SOL 581 | | | | | | x | x | | x | | | x |
| UV SOL 582 | | | | | | x | x | | | x | x | |
| UV SOL 583 | | | | | | x | x | | | x | | x |
| UV SOL 584 | | | | | | x | x | | | | x | x |
| UV SOL 585 | | | | | | | x | x | x | x | | |
| UV SOL 586 | | | | | | | x | x | x | | x | |
| UV SOL 587 | | | | | | | x | x | x | | | x |
| UV SOL 588 | | | | | | | x | x | | x | x | |
| UV SOL 589 | | | | | | | x | x | | x | | x |
| UV SOL 590 | | | | | | | | x | x | | x | x |
| UV SOL 591 | | | | | | | | x | x | x | x | |
| UV SOL 592 | | | | | | | | x | x | x | | x |
| UV SOL 593 | | | | | | | | x | x | | x | x |
| UV SOL 594 | | | | | | | | | x | x | x | x |

Abbreviations

| | | |
|---|---|---|
| BP3 | Benzophenone-3 | 131-57-7 |
| BP4 | Benzophenone-4 | 4065-45-6 |
| 3BC | 3-Benzylidene Camphor | 15087-24-8 |
| BEMT | Bis-Ethylhexyloxyphenol Methoxyphenyl Triazine | 103597-45-1 |
| BMBM | Butyl Methoxydibenzoylmethane | 70356-09-1 |
| DBT | Diethylhexyl Butamido Triazone | 154702-15-5 |
| DTS | Drometrizole Trisiloxane | 155633-54-8 |
| EHT | Ethylhexyl Triazone | 88122-99-0 |
| MBC | Methylbenzylidencamphor | |
| EMC | Ethylhexyl Methoxycinnamate | 36861-47-9 |
| DHHB | Diethylamino Hydroxy Benzoyl Hexyl Benzoate | |

Large amounts of UV filter can advantageously be incorporated, so that the agents according to the invention are oily concentrates of UV filters which can be converted later with desired amounts of water into sunscreen final formulations. The absolute amount of UV filters here depends on the nature chosen for the UV filter, the desired dilution of the sunscreen final formulation and the desired sun protection factor.

Although the agent according to the invention tolerates relatively large amounts of water, in the context of the invention an oily concentrate is desired, so that the amounts of water are preferably as low as possible.

In the context of the invention it is therefore preferable for the agents according to the invention to have no or only small amounts of water of less than 10 wt. %, preferably 0 to 5 wt. %.

With the agents according to the invention it is now possible to achieve sunscreen final formulations having high SPF values, preferably of up to 50+, via the amount and choice of the UV filters incorporated.

The sun protection simulators, for example, of BASF Personal Care and Nutrition GmbH can be used for determining the SPF values.

Finally, the agents according to the invention can comprise water and/or auxiliary substances to 100 wt. %.

Auxiliary substances concentration be, for example, pH adjusters. For example triethanolamine, monoethanolamine or Tetrahydroxypropyl Ethylenediamine. Triethanolamine is preferred.

Agents which are suitable according to the invention are those which comprise
(a) 20 to 35 wt. % of oily substances,
(b) 7 to 17 wt. % of anionic surfactants,
(c) 3 to 18 wt. % of co-surfactants,
(d) 35 to 65 wt. % of UV filters and
(e) 0.01 to 5 wt. % of water and optionally 0.5 to 5 wt. % of auxiliary substances, under the condition that all the constituents add up to 100 wt. %.

Agents which are particularly preferred are those which comprise
(a) 20 to 35 wt. % of an oily substance selected from linear and branched fatty alcohol carbonates, in particular Dicaprylyl Carbonate and/or esters of C₂-C₁₂-dicarboxylic acids
(b) 7 to 17 wt. % of anionic surfactants selected from alkyl polyalkylene glycol ether citrates, in particular the polyethylene glycol ether of lauryl alcohol;
(c) 3 to 18 wt. % of co-surfactants selected from nonionic surfactants, preferably polyol and/or polyglycerol esters
(d) 35 to 65 wt. % of UV filters selected from
   (d₉ₐ) Ethylhexyl Methoxycinnamate
   (d₁₁ₐ) Bis-Ethylhexyloxyphenol Methoxyphenyl Triazine
   (d₁₃ₐ) Ethylhexyl Triazone and
   (d₃ₐ) Diethylamino Hydroxy Benzoyl Hexyl Benzoate; and
(e) 0.01 to 5 wt. % of water and optionally 0.5 to 5 wt. % of auxiliary substances;
wherein
components (d₉ₐ), (d₁₁ₐ), (d₁₃ₐ) and (d₃ₐ) can be present in the agent as an individual compound or a 2-, 3- or 4-component mixture.

The agents according to the invention can be prepared on the laboratory scale by simple, manual stirring at room temperature or, if they comprise a substance which is solid at room temperature, at elevated temperatures. Preferably, the oily substance (a) is initially introduced into the mixing vessel and the UV filters (d) are stirred in, preferably at elevated temperatures and in particular at 85 to 95° C. Clear mixtures are obtained and are preferably cooled to room temperature before the anionic surfactants (b) and co-surfactants (c) and optionally auxiliary substances are stirred in. Additional water which is not incorporated in the form of aqueous formulations of the surfactants and/or auxiliary substances can be added in the last process step, but in the context of the invention is not desired.

In a further preferred preparation procedure, the oily substance (a) is initially introduced into the mixing vessel and the UV filters (d) are stirred in, preferably at elevated temperatures and in particular at 85 to 95° C. Thereafter, the anionic surfactants (b) and the co-surfactants (c) are stirred in. The mixture is then cooled. Auxiliary substances are then optionally also added, such as e.g. pH adjusters or additional alkyl polyglucoside. A clear mixture is obtained by this procedure.

The clear concentrates having a high content of UV filter(s) are furthermore distinguished by a very long shelf life of several weeks.

The present invention also provides a process for the preparation of sunscreen final formulations. The process is characterized in that the agents as claimed in claim 1 are diluted with water and optionally further UV filters and optionally conventional further auxiliary substances at temperatures in the range of from 5 to 30° C.

Advantageously, the dilution is carried out in a so-called cold process, that is to say at room temperature, preferably in the range of from 20 to 25° C., the agents according to the invention being converted into the sunscreen final formulations with water and optionally conventional further auxiliary substances.

The UV filters (d₁)-(d₂₀) already mentioned can be added to the agents according to the invention as further UV filters. Examples of substances are mentioned in the following Table 3:

TABLE 3
Suitable UV filters and auxiliaries which can additionally be used

| | | |
|---|---|---|
| No. | Chemical name | CAS No. |

| | |
|---|---|
| (2-Hydroxy-4-methoxyphenyl)(4-methylphenyl)methanone | 1641-17-4 |
| alpha-(2-Oxoborn-3-ylidene)toluene-4-sulfonic acid and its salts (Mexoryl SL) | 56039-58-8 |
| Methyl N,N,N-trimethyl-4-[(4,7,7-trimethyl-3-oxobicyclo[2,2,1]hept-2- | 52793-97-2 |
| ylidene)methyl]anilinium sulfate (Mexoryl SO) | |
| Menthyl o-aminobenzoate | 134-09-8 |
| 2-Phenyl-1H-benzimidazole-5-sulfonic acid; phenylbenzimidazolesulfonic | 27503-81-7 |
| acid | |
| 2-Propenamide, N-[[4-[(4,7,7-trimethyl-3-oxobicyclo[2.2.1]hept-2- | 147897-12-9 |
| ylidene)methyl]phenyl]methyl]-, homopolymer | |
| 3,3′-(1,4-Phenylenedimethylene)bis[7,7-dimethyl-2-oxo- | 90457-82-2 |
| bicyclo[2.2.1]heptane-1-methanesulfonic acid] (Cibafast H) | |
| 1H-Benzimidazole-4,6-disulfonic acid, 2,2′-(1,4-phenylene)bis-, disodium salt | 180898-37-7 |
| Phenol, 2-(2H-benzotriazol-2-yl)-4-methyl-6-[2-methyl-3-[1,3,3,3-tetramethyl- | 155633-54-8 |
| 1-[(trimethylsilyl)oxy]disiloxanyl]propyl]-; drometrizole trisiloxane (Mexoryl XL) | |
| 1-Dodecanaminium, N-[3-[[4-(dimethylamino)benzoyl]amino]propyl]N,N- | 156679-41-3 |
| dimethyl-, salt with 4-methylbenzenesulfonic acid (1:1) (Escalol HP610) | |
| 1-Propanaminium, N,N,N-trimethyl-3-[(1-oxo-3-phenyl-2-propenyl)amino]-, | 177190-98-6 |
| chloride | |
| 1H-Benzimidazole-4,6-disulfonic acid, 2,2′-(1,4-phenylene)bis- | 170864-82-1 |
| 1-Propanaminium, 3-[[3-[3-(2H-benzotriazol-2-yl)-5-(1,1-dimethylethyl)-4- | 340964-15-0 |
| hydroxyphenyl]-1-oxopropyl]amino]-N,N-diethyl-N-methyl-, methyl sulfate | |
| (salt) | |
| 2-Propenoic acid, 3-(1H-imidazol-4-yl)- | 104-98-3 |
| 1,2,3-Propanetriol, 1-(4-aminobenzoate) (Glyceryl PABA) | 136-44-7 |
| Benzene acetic acid, 3,4-dimethoxy-a-oxo- | 4732-70-1 |
| 2-Propenoic acid, 2-cyano-3,3-diphenyl-, ethyl ester | 5232-99-5 |
| Anthralinic acid, p-menth-3-yl ester | 134-09-8 |
| 2,2′-Bis(1,4-phenylene)-1H-benzimidazole-4,6-disulfonic acid monosodium | 349580-12-7 |
| salt or Disodium phenyl dibenzimidazole tetrasulfonate (Neo Heliopan AP) | |
| Benzenesulfonic acid, 5-benzoyl-4-hydroxy-2-methoxy-, sodium salt | 6628-37-1 |

| | | |
|---|---|---|
| Benzoic acid, 4,4′-[[6-[[3-[1,3,3,3- tetramethyl-1-[(trimethylsilyl)oxy]- 1-disiloxanyl]propyl]amino]-1,3,5- triazine-2,4-diyl]diimino]bis-, dibutyl ester | 207562- 42-3 | 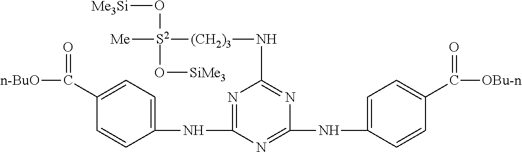 |
| Phenol, 2-(2H-benzotriazol-2-yl)- 6-[[(2-ethylhexyl)oxy]methyl]-4- methyl- | 1314118- 38-1 | 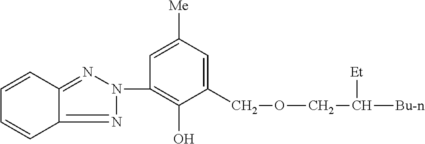 |
| 2-Propenoic acid, 3-(4- ethoxyphenyl)-, 2-methylphenyl ester | 433305- 97-6 | 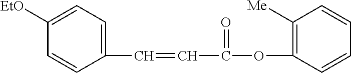 [SMILES: CCOc1ccc(C=CC(=O)Oc2ccccc2C)cc1] |
| 2-Propenoic acid, 3-(4- methoxyphenyl)-, 2-methylphenyl ester | 431067- 87-7 | 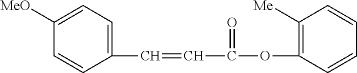 [SMILES: COc1ccc(C=CC(=O)Oc2ccccc2C)cc1] |
| Propanedioic acid, 2-[(4- methoxyphenyl)methylene]-, 1,3- bis(2-methylbutyl) ester | 1257307- 15-5 | 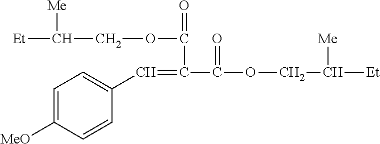 [SMILES: CCC(C)COC(=O)C(=Cc1ccc(OC)cc1)C(=O)OCC(C)CC] |
| Propanedioic acid, 2-[(4- methophenyl)methylene]-, 1,3- bis(2-ethylhexyl) ester | 189183- 15-1 | 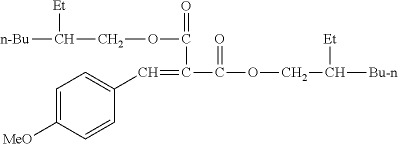 |

Polymeric benzotriazole UV filters as described e.g. in US 20110195036, WO
2011097555; i.e. sunscreen formulations comprising a polymer prepared from a dimeric
diol (36H72O), ditrimethylolpropane, dimethyl adipate, methyl adipate, and methyl 3-(2H-
benzotriazol-2-yl)-5-(1,1-dimethylethyl)-4-hydroxybezenepropanoate

| | | |
|---|---|---|
| 2-Ethoxy-2′-ethyl-oxanilide | CAS No 23949- 66-8 | 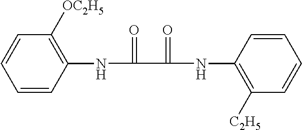 [SMILES: CCOc1ccccc1NC(=O)C(=O)Nc1ccccc1CC] |
| Uvinul S-Pack | CAS | |
| | 852282- | |
| | 89-4 | |

N-(2,6-Diisopropylphenyl)-6-[4-(1,1,3,3-tetramethylbutyl)phenoxy]-1H-
benzo[d,e]isoquinoline-1,3(2H)-dione

| | |
|---|---|
| 1,4-Dihydropyridine derivatives and ionic 1,4-dihydropyridine | 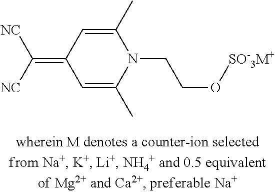 |

Each of the UV filters listed in the above table can be used as an additional filter in the composition according to the invention. One, two, three, four, five or six further UV filters can be employed.

Particularly finely divided and skin-friendly O/W sunscreen final formulations are obtained by the process according to the invention if the highly concentrated agents according to the invention are homogenized in amounts of from 6 to 50 wt. %, preferably >10 to 50 wt. %, and in particular from 15 to 50 wt. %, based on the O/W sunscreen final formulation, with water and optionally auxiliaries.

The homogenization is preferably carried out under moderate mechanical action, that is to say by simple stirring. Particular shearing forces are possible, but not necessary.

It has proved advantageous for thickeners already to be present as further auxiliaries during the dilution with water. Suitable thickeners are anionic, zwitter-ionic, amphoteric and nonionic copolymers, such as, for example, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and esters thereof, acrylamidopropyltrimethylammonium chloride/acrylate copolymers, octylacrylamide/methyl methacrylate/tert-butylaminoethyl methacrylate/2-hydroxypropyl methacrylate polymers, vinylpyrrolidone/vinyl acetate copolymers, vinylpyrrolidone/dimethylaminoethyl methacrylate/vinylcaprolactam terpolymers and optionally polysaccharides, in particular xanthan gum, guar and guar derivatives, agar-agar, alginates and tyloses, cellulose and cellulose derivatives, such as carboxymethylcellulose, carboxymethylcellulose and hydroxycellulose and moreover silicones. Preferably, thickeners from the group of polyacrylates and crosslinked polyacrylates, such as Rheocare TTA®, Cosmedia® SP, Rheocare® C Plus, Tinovis® ADE, Tinovis® GTC, are added, and in particular preferably in amounts of from 0.5 to 5, in particular from 1. to 4 wt. %, calculated as active substance and based on the sunscreen final formulation. Thickeners from the group of polysaccharides, such as Keltrol® T or Rheocare® XG, are furthermore preferred.

The thickeners can be added to the concentrated agent before the dilution with water is carried out or can be contained in the water with which the dilution of the concentrated agent is carried out.

According to a preferred process variant, the concentrated agent is mixed with the thickener, and water for dilution is added to this mixture and the further formulation constituents are optionally stirred in.

According to another preferred process variant, the water, the thickener and optionally the other auxiliary substances are stirred with one another and the concentrated agent is added to this mixture.

The sunscreen final formulations prepared by the process according to the invention are often particularly finely divided O/W emulsion having an average particle size of <10 μm, preferably <5 μm.

The present invention also provides the use of the concentrated agents as claimed in claim 1 for the preparation of cosmetic formulations having a very high sun protection factor, preferably having an SPF of up to 50+.

If desired, the sunscreen final formulations can comprise further auxiliary substances, such as moisture-retaining agents/skin-moisturizing agents, viscosity regulators, oils, fats and waxes, surfactants, pearlescent waxes, super-oiling agents, stabilizers, cationic, zwitter-ionic or amphoteric polymers, further UV filters, biogenic active compounds, film-forming agents, swelling agents, hydrotropic substances, preservatives, solubilizers, perfume oils, dyestuffs, insect repellant active compounds etc., which are listed below by way of example.

Moisture-retaining agents serve to further optimize the sensory properties of the composition and for moisture regulation of the skin. The moisture-retaining agents can be present in an amount of 0-5 wt. %. Suitable substances are, inter alia, amino acids, pyrrolidonecarboxylic acid, lactic acid and salts thereof, lactitol, urea and urea derivative, uric acid, glucosamine, creatinine, collagen cleavage products, chitosan or chitosan salts/derivatives, and in particular polyols and polyol derivatives (e.g. glycerol, diglycerol, triglycerol, ethylene glycol, propylene glycol, butylene glycol, erythritol, 1,2,6-hexanetriol, polyethylene glycols, such as PEG-4, PEG-6, PEG-7, PEG-8, PEG-9, PEG-10, PEG-12, PEG-14, PEG-16, PEG-18, PEG-20), sugars and sugar derivatives (inter alia fructose, glucose, maltose, maltitol, mannitol, inositol, sorbitol, sucrose, sorbitylsilanediol, sucrose, trehalose, xylose, xylitol, glucuronic acid and salts thereof), ethoxylated sorbitol (sorbeth-6, sorbeth-20, sorbeth-30, sorbeth-40), honey and hardened honey, hardened starch hydrolysates and mixtures of hardened wheat protein and PEG-20/acetate copolymer. Substances which are preferably suitable according to the invention as moisture-retaining agents are glycerol, diglycerol, triglycerol and butylene glycol.

Possible insect repellants are, for example, N,N-diethyl-m-toluamide, 1,2-pentanediol or 3-(N-n-butyl-N-acetylamino)propionic acid ethyl ester), which is marketed by Merck KGaA under the name Insect Repellent 3535, and butylacetylaminoproprionate. They are conventionally employed in the compositions according to the invention in an amount of 0-6 wt. %, based on the agent.

The viscosity of the agents according to the invention can be achieved by addition of viscosity regulators. Possible viscosity regulators are, inter alia, agents which impart consistency, such as e.g. fatty alcohols or hydroxy-fatty alcohols having 12 to 22 and preferably 16 to 18 carbon atoms and partial glycerides, fatty acids having 12 to 22 carbon atoms or 12-hydroxy-fatty acids. A combination of these substances with alkyl oligoglucosides and/or fatty acid N-methylglucamides of the same chain length is also suitable, since such combinations deliver particularly stable and homogeneous emulsions. The viscosity regulators also include thickening agents, such as, for example, Aerosil types (hydrophilic silicic acids), polysaccharides, in particular xanthan gum, guar-guar, agar-agar, alginates and tyloses, carboxymethylcellulose and hydroxyethyl- and hydroxypropylcellulose, furthermore higher molecular weight polyethylene glycol mono- and diesters of fatty acids, polyacrylates (e.g. Carbopols® and Pemulen types from Goodrich; Synthalens® from Sigma; Keltrol types from Kelco; Sepigel types from Seppic; Salcare types from Allied Colloids), non-crosslinked and polyol-crosslinked polyacrylic acids, polyacrylamides, polyvinyl alcohol and polyvinylpyrrolidone. Bentonites, such as e.g. Bentone® Gel VS-5PC (Rheox), which is a mixture of cyclopentasiloxane, Disteardimonium Hectorite and propylene carbonate, have also proved to be particularly effective. Surfactants, such as, for example, ethoxylated fatty acid glycerides, esters of fatty acids with polyols, such as, for example, pentaerythritol or trimethylolpropane, fatty alcohol ethoxylates with a narrowed homologue distribution, alkyl oligoglucosides and electrolytes, such as e.g. sodium chloride and ammonium chloride, can also be employed for regulation of the viscosity.

In the context of the invention fats and waxes are understood as meaning all lipids having a fat- or wax-like consistency which have a melting point above 20° C. These include, for example, the classic triacylglycerols, that is to say the triesters of fatty acids with glycerol, which can be of plant or animal origin. These can also be mixed esters, that is to say triesters of glycerol with various fatty acids, or a mixture of various glycerides. These also include mixtures of mono-, di- and triglycerides. So-called hardened fats and oils which are obtained by partial hydrogenation are particularly suitable according to the invention. Hardened fats and oils of plants are preferred, e.g. hydrogenated castor oil, groundnut oil, soya oil, rape oil, beet seed oil, cottonseed oil, soya oil, sunflower oil, palm oil, palm kernel oil, linseed oil, almond oil, maize oil, olive oil, sesame oil, cacao butter and coconut fat. Oxidation-stable plant glycerides which are available under the name Cegesoft® or Novata® are particularly suitable.

Possible waxes are, inter alia, natural waxes, such as e.g. candelilla wax, carnauba wax, Japan wax, esparto grass wax, cork wax, guaruma wax, rice germ oil wax, sugar cane wax, ouricury wax, montan wax, beeswax, shellac wax, spermaceti, lanolin (wool wax), uropygium fat, ceresin, ozocerite (earth wax), petrolatum, paraffin waxes, microwaxes; chemically modified waxes (hard waxes), such as e.g. montan ester waxes, Sasol waxes, hydrogenated jojoba waxes and synthetic waxes, such as e.g. polyalkylene waxes and polyethylene glycol waxes.

In addition to the fats, fat-like substances, such as lecithins and phospholipids, are also possible as additives. Lecithins are glycero-phospholipids which are formed from fatty acids, glycerol, phosphoric acid choline by esterification, and are often also called phosphatidylcholines (PC). Cephalins, which are also called phosphatidic acids and are derivatives of 1,2-diacyl-sn-glycerol-3-phosphoric acids, may be mentioned as an example of natural lecithins. In contrast, phospholipids are usually understood as meaning mono- and preferably diesters of phosphoric acid with glycerol (glycerol phosphates). Sphingosines and sphingolipids are also possible as fat-like substances.

Suitable pearlescent waxes are, for example, alkylene glycol esters, specifically ethylene glycol distearate; fatty acid alkanolamides, specifically coconut fatty acid diethanolamide; partial glycerides, specifically stearic acid monoglyceride; esters of polybasic, optionally hydroxy-substituted carboxylic acids with C₆-C₂₂-fatty alcohols, specifically long-chain esters of tartaric acid; fatty substances, such as, for example, fatty alcohols, fatty ketones, fatty aldehydes, fatty ethers and fatty carbonates, which have at least 24 carbon atoms in total—specifically Lauron®; distearyl ether; fatty acids, such as stearic acid, C₁₂-C₂₂-hydroxy-fatty acids, behenic acid, ring-opening products of C₁₂-C₂₂-olefin epoxides with C₁₂-C₂₂-fatty alcohols and/or polyols having 2 to 15 carbon atoms and 2 to 10 hydroxyl groups and mixtures thereof.

Super-oiling agents which can be used are substances such as, for example, lanolin and lecithin and polyethoxylated or acylated derivatives of lanolin and lecithin, polyol fatty acid esters, monoglycerides and fatty acid alkanolamides, the latter simultaneously serving as foam stabilizers.

So-called stabilizers which can be employed are metal salts of fatty acids, such as e.g. magnesium, aluminum and/or zinc stearate or ricinoleate.

Suitable cationic polymers which further optimize the sensory properties of the compositions according to the invention and impart to the skin a sensation of softness are, for example, cationic cellulose derivatives, such as e.g. a quaternized hydroxyethylcellulose which is obtainable from Amerchol under the name Polymer JR 400®, cationic starch, copolymers of diallylammonium salts and acrylamides, quaternized vinylpyrrolidone/vinylimidazole polymers, such as e.g. Luviquat® (BASF), condensation products of polyglycols and amines, quaternized collagen polypeptides, such as, for example, Lauryldimonium Hydroxypropyl Hydrolyzed Collagen (Lamequat®L/Grunau), quaternized wheat polypeptides, polyethylenimine, cationic silicone polymers, such as e.g. amodimethicone, copolymers of adipic acid and dimethylaminohydroxypropyldiethylenetriamine (Cartaretine®/Sandoz), copolymers of acrylic acid with dimethyldiallylammonium chloride (Merquat® 550/Chemviron, polyaminopolyamides and crosslinked water-soluble polymers thereof, cationic chitin derivatives, such as, for example, quaternized chitosan, condensation products, optionally distributed in microcrystalline form, of dihaloalkyls, such as e.g. dibromobutane with bisdialkylamines, such as e.g. bis-dimethylamino-1,3-propane, cationic guar gum, such as e.g. Jaguar® CBS, Jaguar® C-17, Jaguar® C-16 from Celanese, quaternized ammonium salt polymers, such as e.g. Mirapol® A-15, Mirapol®AD-1, Mirapol® AZ-1 from Miranol.

Starch derivative can furthermore be employed to improve the skin sensation, e.g. Dry Flo® PC (INCI: Aluminum Starch Octenylsuccinate).

Suitable silicone compounds have already been mentioned with the oily substances. In addition to dimethylpolysiloxanes, methylphenylpolysiloxanes and cyclic silicones, amino-, fatty acid-, alcohol-, polyether-, epoxy-, fluorine-, glycoside- and/or alkyl-modified silicone compounds, which can be either liquid or resinous at room temperature, are also suitable. Simethicones, which are mixtures of dimethicones having an average chain length of from 200 to 300 dimethylsiloxane units and silicon dioxide or hydrogenated silicates, are furthermore suitable.

Biogenic active compounds which are suitable according to the invention are to be understood as meaning, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, such as e.g. Prunus extract, Bambara nut extract and vitamin complexes. Such active compounds are employed in sunscreen final formulations as agents which trap free radicals, and serve to regenerate the skin.

So-called film-forming agents which lead to a further improvement in the sensory properties of the preparations according to the invention are, for example, chitosan, microcrystalline chitosan, quaternized chitosan, collagen, hyaluronic acid and salts thereof and similar compounds, and the polyvinylpyrrolidones, vinyl pyrrolidone/vinyl acetate copolymers, polymers of the acrylic acid series and quaternized cellulose derivatives already mentioned under the viscosity regulators.

To improve the flow properties of the compositions according to the invention hydrotropic substances, such as, for example, ethanol, isopropyl alcohol, or polyols, can furthermore be employed. Polyols which are possible here have preferably 2 to 15 carbon atoms and at least two hydroxyl groups. The polyols can also contain further functional groups, in particular amino groups, or can be modified with nitrogen.

Suitable preservatives are, for example, phenoxyethanol, formaldehyde solution, parabens, pentanediol or sorbic acid and the silver complexes known by the name Surfacine®.

Perfume oils which may be mentioned are natural, plant and animal as well as synthetic odoriferous substances or mixtures thereof. Natural odoriferous substances are obtained, inter alia, by extraction of flowers, stems, leaves, fruit, fruit peel, roots and resins of plants. Animal raw materials are furthermore possible, such as, for example, civet and castoreum. Typical synthetic odoriferous compounds are products of the ester, ether, aldehyde, ketone, alcohol and hydrocarbon type. Preferably, mixtures of various odoriferous substances which together generate a pleasant fragrance note are used.

Dyestuffs which can be used are the substances which are suitable and approved for cosmetic purposes.

The following examples serve to illustrate the invention, without limiting this to the examples.

### EXAMPLES A1 TO A7

### Concentrates Comprising UV Filters

Data calculated as active substance in wt. %

| | C** | A1 | A2 | A3 | A4 | Com. 1 | Com. 2 | Com. 3 |
|---|---|---|---|---|---|---|---|---|
| Polyglyceryl-2 | (c) | 10.0 | 10.0 | 10.0 | 17.0 | — | 10 | — |
| Dipolyhydroxystearate | | | | | | | | |
| Dicaprylyl Carbonate | (a) | 23.9 | — | — | — | — | — | — |
| Dibutyl Adipate | (a) | — | 23.9 | 27 | 20.0 | 23.8 | 23.8 | 23.8 |
| Laureth-7 Citrate* | (b) | 15.0 | 15.0 | 12.0 | 12.0 | 15.0 | — | — |
| NaOH (as a 50 wt. % solution)) | | 0.1 | 0.1 | — | — | 0.0 | 0.0 | 0.0 |
| Ethylhexyl Methoxycinnamate | (d) | 20.0 | 20.0 | 20.0 | 20.0 | 20.0 | 20.0 | 20.0 |
| Bis-Ethylhexyloxyphenol | | 5.0 | 5.0 | 5.0 | 5.0 | 5.0 | 5.0 | 5.0 |
| Methoxyphenyl Triazine | | | | | | | | |
| Ethylhexyl Triazone | | 6.0 | 6.0 | 6.0 | 6.0 | 6.0 | 6.0 | 6.0 |
| Diethylamino Hydroxybenzoyl | | 20.0 | 20.0 | 20.0 | 20.0 | 20.0 | 20.0 | 20.0 |
| Hexyl Benzoate | | | | | | | | |
| Appearance (1 day) | | clear | clear | clear | clear | cloudy | cloudy | clear |
| | | single | single | single | single | | | |
| | | phase | phase | phase | phase | | | |

*Water content of Laureth-7 Citrate less than 0.5 wt. % (approx. 0.3 wt. %)
**Component according to the invention

The concentrates according to the invention are prepared by mixing the oily component (a) (Dicaprylyl Carbonate or Dibutyl Adipate) with the UV filters (d) (Ethylhexyl Methoxycinnamate, Bis-Ethylhexyloxyphenol Methoxyphenyl Triazine, Ethylhexyl Triazone, Diethylamino Hydroxybenzoyl Hexyl Benzoate), while heating to 90 to 95° C. After a clear solution is obtained this is cooled to room temperature and the anionic surfactant (b) (Laureth-7 Citrate) and the co-surfactant (c) (Polyglyceryl-2 Dipolyhydroxystearate) are stirred in.

Storage-stable concentrates which even after storage for several weeks at room temperature show no sediment or formation of crystals of the UV filter are obtained.

As a comparison, corresponding concentrates are prepared, but without anionic surfactant (b) (Laureth-7 Citrate) according to Com. 2 or without co-surfactant (c) (Polyglyceryl-2 Dipolyhydroxystearate) according to Com. 1 or completely without surfactants according to Com. 3. The concentrate according to Com. 1 is cloudy, the concentrate according to Com. 2 is slightly cloudy. The concentrate according to Com. 3 forms undesirable crystals during storage within 4 weeks. Furthermore, the concentrate according to Com. 3 is not self-emulsifying on dilution with water.

### EXAMPLES A6-A9

### Further Examples of Concentrates (Data in Wt. % of Product)

| INCI name | C * | A6 | A7 | A8 | A9 |
|---|---|---|---|---|---|
| Polyglyceryl-2 Dipolyhydroxystearate | (c) | 8 | 8 | 8 | 7.5 |
| Dicaprylyl Carbonate | (a) | 0 | 0 | 0 | 0 |
| Dibutyl Adipate | | 28.75 | 28.0 | 28.0 | 28.0 |
| Laureth-7 Citrate | (b) | 12.25 | 9.0 | 9.0 | 11.5 |
| NaOH (as a 50 wt. % solution)) | | 0 | 0 | 0 | 0 |
| Ethylhexyl Methoxycinnamate | (d) | 20 | 20 | 20 | 20 |
| Bis-Ethylhexyloxyphenol Methoxyphenyl Triazine | | 5 | 5 | 5 | 5 |
| Ethylhexyl Triazone | | 6 | 6 | 6 | 6 |
| Diethylamino Hydroxybenzoyl Hexyl Benzoate | | 20 | 20 | 20 | 20 |
| Lauryl Glucoside | | 0 | 2 | 3 | 0 |
| Triethanolamine | | 0 | 2.0 | 1.0 | 2.0 |

| | | | | |
|---|---|---|---|---|
| Appearance (1 day) | clear | clear | clear | clear |
| | single phase | single phase | single phase | single phase |

** Component according to the invention

### EXAMPLES B1 to B3

### O/W Sunscreen Final Formulations Based on the Concentrates Comprising UV Filters (Data Calculated as Active Substance in Wt. %)

| Compound | B1 | B2 | B3 | Com. B |
|---|---|---|---|---|
| Rheocare TTA ® | 3.5 | | — | — |
| Acrylates copolymer (thickener) | | | | |
| Cosmedia ® SP | | 1.5 | — | — |
| Sodium Polyacrylate | | | | |
| (thickener) | | | | |
| Concentrate according to Example A3 | 30 | 30 | | — |
| Concentrate according to Example A4 | | | 10 | |
| Concentrate according to Com. 3 | | | | 10 |
| Glycerol | — | 5.0 | | — |
| Magnesium sulfate (solid) | — | 0.5 | | — |
| NaOH (as a 50 wt. % solution)) | qs | qs | | — |
| Preservative | qs | qs | qs | — |
| Aqua dem. | to 100 | to 100 | to 100 | to 100 |
| Appearance (1 day) | white | white | white | phase |
| | | | | sep. |
| Structure | O/W | O/W | O/W | demixed |
| | emulsion | emulsion | emulsion | |
| Particle diameter | <5 μm | <5 μm | <5 μm | — |
| SPF calc | 30.2 | 30.2 | 9.8 | |
| | ratio 2.44 | ratio 2.44 | ratio 1.81 | |

For sunscreen composition B1 the thickener is homogenized with water and all the other constituents apart from the concentrate and the mixture is introduced into the mixing vessel. The concentrate according to Example A3 is then added to this and all the components are homogenized with the magnetic stirrer. The pH is adjusted to 6.5.

For sunscreen composition B2 the thickener is mixed with the concentrate according to Example A3 and the mixture is introduced into the mixing vessel. Water is then added as well as the remainder of the recipe substances and all the components are homogenized with the magnetic stirrer. The pH is adjusted to 5.0.

For sunscreen composition B3, which comprises no thickener, water and the remainder of the recipe substances are added and all the components are homogenized with the magnetic stirrer. The pH is adjusted to 5.0.

For comparison purposes the concentrates from Table 1 Com. 1, Com. 2 and Com. 3 are dispersed in water in order to test the self-emulsifying properties. While finely divided 01W emulsions having a particle diameter in the region of less than 5 μm are formed with the concentrates according to the invention, the dispersions with the comparison concentrates are unstable and a distinct phase separation occurs after one day. Com. 3 in particular forms no emulsion in the dilution with water.

### EXAMPLES C1-C14

### Further Recipe Examples for the Sunscreen Products

| | | Example | | | |
|---|---|---|---|---|---|
| Compound | INCI name | C1 | C2 | C4 | C4 |
| Cosmedia ® SP (thickener) | Sodium Polyacrylate | 2.0 | 2.0 | 2.0 | 2.0 |
| Concentrate according to Example 6 | | 50.0 | 30.0 | 15.0 | 8.0 |
| EDTA BD | Disodium EDTA | 0.2 | 0.2 | 0.2 | 0.2 |
| NaOH | Aqua, Sodium hydroxide | | | | |
| (as a 30 wt. % solution) | | | | | |
| Phenonip | Phenoxyethanol/Methyl- | 1.0 | 1.0 | 1.0 | 1.0 |
| (preservative) | paraben/Ethylparaben/ | | | | |
| | Butylparaben/Propylparaben/ | | | | |
| | Isobutylparaben | | | | |

| Aqua dem. | | to 100 | to 100 | to 100 | to 100 |
|---|---|---|---|---|---|
| Appearance (1 day) | | white | white | white | white |
| Structure | | O/W | O/W | O/W | O/W |
| | | emulsion | emulsion | emulsion | emulsion |
| SPF calculated | | 50.2 | 30.2 | 14.8 | 7.9 |
| | | ratio | ratio | ratio | ratio |
| | | 2.61 | 2.44 | 2.01 | 1.7 |
| | | Example | | | |
| Compound | INCI name | C5 | C6 | C7 | C8 |
| Tinovis ADE ® | Sodium Acrylates Copolymer | 2.0 | 2.5 | | |
| (thickener) | Hydrogenated Polydecene | | | | |
| | PPG-1 Trideceth-6-Hydrogenated | | | | |
| | Polydecene PPG-1 | | | | |
| | Trideceth-6 | | | | |
| Tinovis GTC ® | Acrylates/Beheneth-25 Meth- | | | | |
| (thickener) | acrylate Copolymer | | | | |
| Aristoflex HMB ® | Ammonium | | | 0.8 | 0.8 |
| (thickener) | Acryloyldimethyltaurate/Beheneth- | | | | |
| | 25 Methacrylate | | | | |
| | Crosspolymer | | | | |
| Concentrate according to Example 6 | | 50.0 | 8.0 | 50.0 | 6.0 |
| EDTA BD | Disodium EDTA | 0.2 | 0.2 | 0.20 | 0.20 |
| NaOH | Aqua, Sodium hydroxide | 3.3 | 0.90 | 2.70 | 0.35 |
| (as a 30 wt. % solution) | | | | | |
| Phenonip | Phenoxyethanol/Methyl- | 1.0 | 1.0 | 1.0 | 1.0 |
| (preservative) | paraben/Ethylparaben/Butyl- | | | | |
| | paraben/Propylparaben/Iso- | | | | |
| | butylparaben | | | | |

| | | | | |
|---|---|---|---|---|
| Aqua dem. | to 100 | to 100 | to 100 | to 100 |
| Appearance (1 day) | white | white | white | white |
| Structure | O/W | O/W | O/W | O/W |
| | emulsion | emulsion | emulsion | emulsion |
| SPF calculated | 50.2 | 7.9 | 50.2 | 6.1 |
| | ratio | ratio | ratio | ratio |
| | 2.61 | 1.7 | 2.61 | 1.59 |

| | | Example | | | |
|---|---|---|---|---|---|
| Compound | INCI name | C9 | C10 | C11 | C12 |
| Rheocare XG ® (thickener) | Xanthan Gum | 0.5 | 0.5 | | |
| Tinovis GTC ® (thickener) | Acrylates/Beheneth-25 | | | 3.0 | 3.0 |
| | Methacrylate Copolymer | | | | |
| Rheocare C Plus (thickener) | Carbomer | | | | |

| | | | | |
|---|---|---|---|---|
| Concentrate according to Example 6 | 6.0 | 50.0 | 50.0 | 6.0 |

| | | | | | |
|---|---|---|---|---|---|
| EDTA BD | Disodium EDTA | 0.2 | 0.2 | 0.2 | 0.2 |
| NaOH (as a 30 wt. % solution) | Aqua, Sodium hydroxide | | | 3.1 | 1.02 |
| Citric Acid (30%) | Aqua, Citric acid | | | 0.40 | |
| Phenonip | Phenoxyethanol/Methyl- | 1.0 | 1.0 | 1.0 | 1.0 |
| Preservative | paraben/Ethylparaben/ | | | | |
| | Butylparaben/Propylparaben/ | | | | |
| | Isobutylparaben | | | | |

| | | | | |
|---|---|---|---|---|
| Aqua dem. | to 100 | to 100 | to 100 | to 100 |
| Appearance (1 day) | white | white | white | white |
| Structure | O/W | O/W | O/W | O/W |
| | emulsion | emulsion | emulsion | emulsion |
| SPF calculated | 6.1 | 50.2 | 50.2 | 6.1 |
| | ratio | ratio | ratio | ratio |
| | 1.59 | 2.61 | 2.61 | 1.59 |

Example

| Compound | INCI name | C13 | C14 |
|---|---|---|---|
| Rheocare XG ® (thickener) | Xanthan Gum | | |
| Tinovis GTC ® (thickener) | Acrylates/Beheneth-25 | | |
| | Methacrylate Copolymer | | |
| Rheocare C Plus (thickener) | Carbomer | 1.50 | 0.80 |

| | | |
|---|---|---|
| Concentrate according to Example 6 | 50.0 | 6.0 |

| | | | |
|---|---|---|---|
| EDTA BD | Disodium EDTA | 0.2 | 0.2 |
| NaOH (as a 30 wt. % solution) | Aqua, Sodium hydroxide | 5.6 | 1.50 |
| Citric Acid (30%) | Aqua, Citric acid | 2.4 | |
| Phenonip (preservative) | Phenoxyethanol/Methyl- | 1.0 | 1.0 |
| | paraben/Ethylparaben/ | | |
| | Butylparaben/Propylparaben/ | | |
| | Isobutylparaben | | |

| | | |
|---|---|---|
| Aqua dem. | to 100 | to 100 |
| Appearance (1 day) | white | white |
| Structure | O/W | O/W |
| | emulsion | emulsion |
| SPF calculated | 50.2 | 6.1 |
| | ratio | ratio |
| | 2.61 | 1.59 |

| | | Example | | | |
|---|---|---|---|---|---|
| Compound | INCI | C15 | C16 | C17 | C18 |
| EUMULGIN PRISMA | Disodium Cetearyl | | | | |
| | Sulfosuccinate | | | | |
| EUMULGIN SG | Sodium Stearoyl Glutamate | | | | |
| AMPHISOL K | Potassium Cetyl Phosphate | | | | |
| CETIOL CC | Dicaprylyl Carbonate | 5.00 | | | |
| Concentrate according to Example 6 | | 50.00 | 50.00 | 50.00 | 50.00 |
| NaOH 30% | Aqua, Sodium hydroxide | 2.62 | 3.00 | 3.00 | 3.10 |
| Trisamino solution (30%) | Aqua, Thromethamine | | | | |
| EDTA BD | Disodium EDTA | 0.20 | 0.20 | 0.20 | 0.20 |
| TINOVIS ADE | Sodium Acrylates Copolymer/ | 2.60 | 2.50 | 2.60 | 2.50 |
| | Hydrogenated Polydecene/ | | | | |
| | PPG-1 Trideceth-6 | | | | |
| RHEOCARE XG | Xanthan Gum | | | | |
| DC246 | Cyclopentasiloxane | | | | |
| DRY FLO PC | Aluminum Starch | | | | |
| | Octenylsuccinate | | | | |
| EUSOLEX 232 | Phenylbenzimidazole Sulfonic | | | | |
| | Acid | | | | |
| TRIS AMINO ULTRA PURE | Thromethamine | | | | |
| NaOH 30% | Aqua, Sodium hydroxide | | | | |
| Citric acid 30% | Aqua, Citric acid | | | | |
| TINOSORB ®M | Methylene Bis-Benzotriazolyl | | 5.00 | | |
| | Tetramethylbutylphenol | | | | |
| TINOSORB ®A2B | Tris-Biphenyl Triazine | | | 5.00 | |
| TINOSORB ®S-AQUA | Bis-Ethylhexyloxyphenol | | | | 5.00 |
| | Methoxyphenyl Triazine | | | | |
| | Polymethyl Methacrylate | | | | |
| PHENONIP | Phenoxyethanol/Methylparaben/ | 1.00 | 1.00 | 1.00 | 1.00 |
| | Ethylparaben/Butylparaben/ | | | | |
| | Propylparaben/Isobutylparaben | | | | |

| | | | | |
|---|---|---|---|---|
| Aqua dem. | to 100 | to 100 | to 100 | to 100 |
| Appearance (1 day) | white | white | white | white |
| Structure | O/W | O/W | O/W | O/W |
| | emulsion | emulsion | emulsion | emulsion |
| SPF calculated | 50.2 | 60.2 | 61.9 | 57.7 |
| | ratio | ratio | ratio | ratio |
| | 2.61 | 2.59 | 2.59 | 2.75 |

| | | Example | | | | |
|---|---|---|---|---|---|---|
| Compound | INCI | C19 | C20 | C21 | C22 | C23 |
| EUMULGIN PRISMA | Disodium Cetearyl | | | 1.50 | | |
| | Sulfosuccinate | | | | | |
| EUMULGIN SG | Sodium Stearoyl | | | | 1.50 | |
| | Glutamate | | | | | |
| AMPHISOL K | Potassium cetyl | | | | | 1.50 |
| | Phosphate | | | | | |
| CETIOL CC | Dicaprylyl Carbonate | | 5.00 | 5.00 | 5.00 | 5.00 |
| Concentrate according to Exampe 6 | | 50.00 | 50.00 | 50.00 | 50.00 | 50.00 |
| NaOH 30% | Aqua, Sodium hydroxide | | 2.90 | 2.30 | 2.25 | 2.50 |
| Trisamino solution (30%): | Aqua,Tromethamine | 6.50 | | | | |
| EDTA BD | Disodium EDTA | 0.20 | 0.20 | 0.20 | 0.20 | 0.20 |
| TINOVIS ADE | Sodium Acrylates Co- | 2.50 | 1.50 | 1.50 | 0.90 | 0.65 |
| | polymer/Hydrogenated | | | | | |
| | Polydecene/PPG-1 | | | | | |
| | Trideceth-6 | | | | | |
| RHEOCARE XG | Xanthan Gum | | | | | |
| DC246 | Cyclopentasiloxane | | 2.00 | 2.00 | 2.00 | 2.00 |
| DRY FLO PC | Aluminum Starch | | 2.00 | 2.00 | 2.00 | 2.00 |
| | Octenylsuccinate | | | | | |
| EUSOLEX 232 | Phenylbenzimidazole | 2.00 | | | | |
| | Sulfonic Acid | | | | | |
| TRIS AMINO ULTRA PURE | Thromethamine | 0.90 | | | | |
| NaOH 30% | Aqua, Sodium hydroxide | 0.90 | | | | |
| Citric acid 30% | Aqua, Citric acid | | | | | |
| TINOSORB ®M | Methylene Bis- | | 5.00 | 5.00 | 5.00 | 5.00 |
| | Benzotriazolyl | | | | | |
| | Tetramethylbutylphenol | | | | | |
| TINOSORB ®A2B | Tris-Biphenyl Triazine | | | | | |
| TINOSORB ®S-AQUA | Bis-Ethyl hexyloxyphenol | | | | | |
| | Methoxyphenyl Triazine | | | | | |
| | Polymethyl Methacrylate | | | | | |
| PHENON IP | Phenoxyethanol/Methyl- | 1.00 | 1.00 | 1.00 | 1.00 | 1.00 |
| | paraben/Ethylparaben/ | | | | | |
| | Butylparaben/ | | | | | |
| | Propylparaben | | | | | |

| | | | | | |
|---|---|---|---|---|---|
| Aqua dem. | to 100 | to 100 | to 100 | to 100 | to 100 |
| Appearance (1 day) | white | white | white | white | white |
| Structure | O/W | O/W | O/W | O/W | O/W |
| | emulsion | emulsion | emulsion | emulsion | emulsion |
| SPF calculated | 59.9 | 60.2 | 60.2 | 60.2 | 60.2 |
| | ratio | ratio | ratio | ratio | ratio |
| | 3.12 | 2.59 | 2.59 | 2.59 | 2.59 |

| | | | |
|---|---|---|---|
| Phase | Trade name | INCI | wt. % |

Example C24: SP15

| A | Concentrate according to Example 6 | | 15.00 |
|---|---|---|---|
| B | Water | Aqua | 79.80 |
| | EDTA BD | Disodium EDTA | 0.20 |
| | TINOVIS ADE | Sodium Acrylates Copolymer/Hydrogenated | 3.20 |
| | | Polydecene/PPG-1 Trideceth-6 | |
| | NaOH 30% | Aqua, Sodium hydroxide | 0.80 |
| C | PHENONIP | Phenoxyethanol/Methylparaben/Ethylparaben/ | 1.00 |
| | | Butylparaben/Propylparaben | |

| | |
|---|---|
| SPF calculated | 15.00 |

Example C25: SP 50+

| A | CETIOL CC | Dicaprylyl Carbonate | 5.00 |
|---|---|---|---|
| | Concentrate according to Example 6 | | 50.00 |
| | NaOH 30% | Aqua, Sodium hydroxide | 2.50 |
| B | Water | Aqua | 29.80 |
| | EDTA BD | Disodium EDTA | 0.20 |
| | TINOVIS ADE | Sodium Acrylates Copolymer/Hydrogenated | 2.50 |
| | | Polydecene/PPG-1 Trideceth-6 | |
| C | DC246 | Cyclopentasiloxane | 2.00 |
| | DRY FLO PC | Aluminum Starch Octenylsuccinate | 2.00 |
| D | TINOSORB ®M | Methylene Bis-Benzotriazolyl | 5.00 |
| | | Tetramethylbutylphenol | |
| E | PHENONIP | Phenoxyethanol/Methylparaben/Ethylparaben/ | 1.00 |
| | | Butylparaben/Propylparaben | |

| | |
|---|---|
| Perfume Jojoba oil | |
| SPF | 57.50 |

Example C26: BB Cream

| A | Concentrate according to Example 6 | | 30.00 |
|---|---|---|---|
| B | Water | Aqua | |
| | Eumulgin BA25 | Beheneth-25 | 1.00 |
| | RHEOCARE XG | Xanthan Gum | 0.50 |
| | BUTYLENE GLYCOL | Butylene Glycol | 5.00 |
| C | DC 245 Fluid | Cyclopentasiloxane | 3.00 |
| D | Chione M-SVA | Synthetic Fluorphlogopite, Lauroyl Lysine | 1.00 |
| | Timica Terra White | Mica, Titanium Dioxide | 1.50 |
| | Timica Terra Yellow | Mica, Titanium Dioxide | 2.00 |
| | Timica Terra Red | Mica, Iron Oxides, Titanium Dioxide | 0.50 |
| | Timica Terra Black | Mica, Iron Oxides, Titanium Dioxide | 0.20 |
| | PHENONIP | PhenoxyethanolMethylparaben/Ethylparaben/ | 1.00 |
| | | Butylparaben/Propylparaben | |

| | |
|---|---|
| Perfume Cabano Rose | 0.20 |
| SPF | 31.00 |

Example C27: Daily

| A | Concentrate according to Example 6 | | 15.00 |
|---|---|---|---|
| | Cetiol Sensoft | Propylheptyl Caprylate | 10.00 |
| B | Water | Aqua | 64.2 |
| | Glycerin 85% | Glycerin | 3.00 |
| | EDTA BD | Disodium EDTA | 0.20 |
| | Cosmedia SP | Sodium Polyacrylate | 0.40 |
| C | Tinovis ADE | Sodium Acrylates Copolymer/Hydrogenated | 1.50 |
| | | Polydecene/PPG-1 Trideceth-6 | |
| D | Orgasol Caresse | Polyamide-5 | |
| | Techpolymer MBP8 | Polymethyl Methacrylate | 2.00 |
| | DC245 | Cyclopentasiloxane | 2.00 |
| | Dermican SPB LS9837 | Glycerin, Aqua, Acetyl Tetrapeptide-9 | 0.50 |
| E | Perfume Cabano Rose | | 0.20 |
| F | PHENONIP | Phenoxyethanol/Methylparaben/Ethylparaben/ | 1.00 |
| | | Butylparaben/Propylparaben | |

| | |
|---|---|
| SPF | 25.00 |

Example C28: BB Cream SPF30

| A | Concentrate according to Example 6 | | 30.00 |
|---|---|---|---|
| | Eumulgin BA25 | Beheneth-25 | 1.00 |
| B | Water | Aqua | 40.10 |
| | RHEOCARE XG | Xanthan Gum | 0.50 |
| | BUTYLENE GLYCOL | Butylene Glycol | 5.00 |
| C | DC 245 Fluid | Cyclopentasiloxane | 0.50 |
| D | Chione M-SVA | Synthetic Fluorphlogopite (and) Lauroyl | 1.00 |
| | | Lysine | |
| | Timica Terra White | Mica, Iron Oxides,Titanium Dioxide | 3.00 |
| | Timica Terra Yellow | Mica, Iron Oxides, Titanium Dioxide | 4.00 |
| | Timica Terra Black | Mica, Iron Oxides,Titanium Dioxide | 0.40 |
| | PHENONIP | Phenoxyethanol/Methylparaben/Ethyl- | 1.00 |
| | | paraben/Butylparaben/Propylparaben | |

Example C29: Powder

| Concentrate according to Example 6 | | 5.00 |
|---|---|---|
| Covi-ox T 90 EU | Tocopherol | 0.10 |
| Cetiol C5 | Coco-Caprylate | 0.50 |
| Cetiol B | Dibutyl Adipate | 0.70 |
| Protectol PE | Phenoxyethanol | 0.50 |
| Potassium sorbate | Potassium Sorbate | 0.30 |
| Boroneige superfine | Boron Nitride | 5.00 |
| Z cote HP1 | Zinc oxide (and) Triethoxycaprylylsilane | 20.00 |
| KSP-411 | Polysilicone-22 | 5.00 |
| Talc | Talc | 16.80 |
| Chione M-SVA | Synthetic fluorphlogopite (and) Lauroyl | 31.50 |
| | lysine | |
| Timica Terra Yellow | Mica, Iron Oxides, Titanium Dioxide | 10.00 |
| Timica Terra Red | Mica, Iron Oxides, Titanium Dioxide | 4.00 |
| Timica Terra Black | Mica, Iron OxidesTitanium Dioxide | 0.60 |

Example C30: WO/SPF 30

| A | Concentrate according to Example 6 | | 50.00 |
|---|---|---|---|
| | Dehymuls PGPH | Polyglyceryl-2 Dipolyhydroxystearate | 3.00 |
| | Lameform TGI | Polyglyceryl-3 Diisostearate | 1.00 |
| | Magnesium stearate | Magnesium Stearate | 1.00 |
| B | Water | Aqua | 35.00 |
| | Magnesium sulfate heptahydrate | Magnesium sulfate | 1.00 |
| | Glycerin 85% | Glycerin | 3.00 |
| C | Xiameter PMX-0345 | Cyclopentasiloxane (and) | 5.00 |
| | | Cyclohexasiloxane | |
| | Phenonip | Phenoxyethanol/Methylparaben/Ethyl- | 1.00 |
| | | paraben/Butylparaben/Propylparaben | |

Example C31: Lipstick

| A | Candelilla Wax | Candelilla Wax | 6.00 |
|---|---|---|---|
| | Carnauba Wax T1 | Carnauba Wax | 5.00 |
| | Performa V 1608 Polymer | C30-3 Olefin/Isopropyl Maleate/MA | 4.00 |
| | | Copolymer | |
| | Performa V 343 | Synthetic Wax | 4.00 |
| | Plandool-G | Bis-Behenyl/Isostearyl/Phytosteryl Dimer | 7.00 |
| | | Dilinoleyl Dimer Dilinoleate | |
| B | Concentrate according to Example 6 | | 50.00 |
| | Nikkol DGTIS | Polyglyceryl-2 Triisostearate | 14.00 |
| | Cetiol SB 45 | Butyrospermum Parkii (Shea Butter) | 10.00 |
| C | Chione Digital pink | Synthetic Fluorphlogopite, Titanium | 1.00 |
| | | Dioxide | |

| | |
|---|---|
| Perfume | 0.50 |

1. An agent for the preparation of sunscreen final formulations, comprising
(a) 15 to 50 wt. % of at least one oily substance,
(b) 4 to 30 wt. % of at least one anionic surfactant,
(c) 0.5 to 25 wt. % of at least one co-surfactant which differs from (b),
(d) 25 to 75 wt. % of at least one UV filter, and
(e) optionally, water and/or auxiliary substances to 100 wt. %.

2. The agent as claimed in claim 1, wherein (a) comprises at least one medium polarity oil.

3. The agent as claimed in claim 1, wherein (a) comprises at least one linear or branched fatty alcohol carbonate(s).

4. The agent as claimed in claim 1, wherein (a) comprises dicaprylyl carbonate or dibutyl adipate.

5. The agent as claimed in claim 1, wherein (a) is present in an amount ranging from 20 to 35 wt. %.

6. The agent as claimed in claim 1, wherein (b) comprises at least one anionic surfactant that is liquid at 18 to 25° C. as (b).

7. The agent as claimed in claim 1, wherein (b) comprises at least one alkyl polyalkylene glycol ether citrate.

8. The agent as claimed in claim 1, wherein (b) is present in an amount ranging from 7 to 17 wt. %.

9. The agent as claimed in claim 1, wherein (c) comprises at least one polyol and/or polyglycerol ester and/or nonionic surfactant.

10. The agent as claimed in claim 1, wherein (d) is selected from the group consisting of at least one:
(d₁) p-aminobenzoic acid derivative(s);
(d₂) salicylic acid derivative(s);
(d₃) benzophenone derivative(s);
(d₄) dibenzoylmethane derivative(s);
(d₅) diphenyl acrylate(s);
(d₆) 3-imidazol-4-yl-acrylic acid and its ester(s);
(d₇) benzofuran derivative(s);
(d₈) polymeric UV absorber(s);
(d₉) cinnamic acid derivative(s);
(d₁₀) camphor derivative(s);
(d₁₁) hydroxyphenyltriazine derivative(s);
(d₁₂) benzotriazole derivatives;
(d₁₃) trianilino-s-triazine derivatives;
(d₁₄) 2-phenylbenzimidazole-5-sulfonic acid and salts thereof;
(d₁₅) menthyl o-aminobenzoates;
(d₁₆) homosalates;
(d₁₇) tris-biphenyl-triazine derivatives;
(d₁₈) TiO₂ (partly encapsulated), ZnO and mica;
(d₁₉) benzylidenemalonates;
(d₂₀) merocyanine derivatives;
(d₂₁) phenylene bis diphenyltriazines;
(d₂₂) imidazoline derivatives; and
(d₂₃) diarylbutadiene derivatives.

11. The agent as claimed in claim 1, wherein (d) comprises at least one oil-soluble UV filter.

12. The agent as claimed in claim 11, wherein (d) is selected from the group consisting of at least one:
(d_{SOL-1}) Benzophenone-3 (BP3);
(d_{SOL-2}) Benzophenone-4 (BP4);
(d_{SOL-3}) 3-Benzylidene camphor (3BC);
(d_{SOL-4}) Bis-Ethylhexyloxyphenol Methoxyphenyl Triazine (BEMT);
(d_{SOL-5}) Butyl Methoxydibenzoylmethane (BMBM);
(d_{SOL-6}) Diethylhexyl Butamido Triazone (DBT);
(d_{SOL-7}) Drometrizole Trisiloxane (DTS);
(d_{SOL-8}) Ethylhexyl Triazone (EHT);
(d_{SOL-9}) Ethylhexyl Methoxycinnamate;
(d_{SOL-10}) Benzylidenemalonate (BM);
(d_{SOL-11}) Diethylamino Hydroxy Benzoyl Hexyl Benzoate (DHHB);
(d_{SOL-12}) Octocrylene;
(d_{SOL-13}) Polysilicone1-15;
(d_{SOL-14}) Homosalate; and
(d_{SOL-15}) Ethylhexyl salicylate.

13. The agent as claimed in claim 11, wherein (d) is selected from the group consisting of at least one:
(d₉ₐ) Ethylhexyl Methoxycinnamate,
(d₁₁ₐ) Bis-Ethylhexyloxyphenol Methoxyphenyl Triazine,
(d₁₃ₐ) Ethylhexyl Triazone and
(d₃ₐ) Diethylamino Hydroxy Benzoyl Hexyl Benzoate.

14. The agent as claimed in claim 1, wherein (d) is present in an amount ranging comprises the UV filters (d) in amounts of from 35 to 65 wt. %.

15. The agent as claimed in claim 1 that comprises 0 to 10 wt % water.

16. The agent as claimed in claim 1 comprising:
(a) 20 to 35 wt. % of at least one oily substance,
(b) 7 to 17 wt. % of at least one anionic surfactant,
(c) 3 to 18 wt. % of at least one co-surfactant,
(d) 35 to 65 wt. % of at least one UV filter and
(e) 0.01 to 5 wt. % of water and optionally 0.5 to 5 wt. % of at least one auxiliary substance,
under the condition that all constituents add up to 100 wt. %.

17. The agent as claimed in claim 16 comprising:
(a) 20 to 35 wt. % a linear and/or branched fatty alcohol carbonates,
(b) 7 to 17 wt. % of at least one alkyl polyalkylene glycol ether citrate(s);
(c) 3 to 18 wt. % of at least one nonionic co-surfactants,
(d) 35 to 65 wt. % of UV filter(s); selected from the group consisting of:
(d₉ₐ) Ethylhexyl Methoxycinnamate,
(d₁₁ₐ) Bis-Ethylhexyloxyphenol Methoxyphenyl Triazine,
(d₁₃ₐ) Ethylhexyl Triazone and
(d₃ₐ) Diethylamino Hydroxy Benzoyl Hexyl Benzoate; and
(e) 0.01 to 5 wt. % of water and optionally 0.5 to 5 wt. % of at least one auxiliary substance;
wherein components (d₉ₐ), (d₁₁ₐ), (d₁₃ₐ) and (d₃ₐ) can be present in the agent as an individual compound or a 2-, 3- or 4-component mixture.

18. A process for the preparation of a sunscreen comprising diluting at least one agent as claimed in claim 1 with water, and optionally with at least one UV filter and optionally at least one conventional auxiliary substance at a temperature ranging from 5 to 30° C.

19. The process as claimed in claim 18, wherein said at least one agent is diluted with water, and/or optionally with at least one thickener, in an amount ranging from 6 to 50 wt % based on the weight of the sunscreen.

20. The process as claimed in claim 19, comprising formulating the sunscreen in the form of an O/W emulsion, W/O emulsion, gel, gel-cream, W/Si emulsion, sprays, creams or lotions.

21. A cosmetic comprising the agent as claimed in claim 1 that has a sun protection factor of up to 50+ and a UVA-PF/SPF of >1/3.
